SEQUENCE LISTING (42 sequences)

SEQ ID NO: 1 - protein, 22 aa, synthetic construct
MLLLVTSLLLCELPHPAFLLIP

SEQ ID NO: 2 - protein, 10 aa, synthetic construct
GGGSSGGGSG

SEQ ID NO: 3 - protein, 12 aa, synthetic construct
ESKYGPPCPPCP

SEQ ID NO: 4 - protein, 12 aa, synthetic construct
ESKYGPPCPSCP

SEQ ID NO: 5 - protein, 22 aa, synthetic construct
ESKYGPPCPPCPGGGSSGGGSG

SEQ ID NO: 6 - protein, 39 aa, synthetic construct
IEVMYPPPYLDNEKSNGTIIHVKGKHLCPSPLFPGPSKP

SEQ ID NO: 7 - protein, 48 aa, synthetic construct
AKPTTTPAPRPPTPAPTIASQPLSLRPEACRPAAGGAVHTRGLDFACD

SEQ ID NO: 8 - protein, 45 aa, synthetic construct
TTTPAPRPPTPAPTIASQPLSLRPEACRPAAGGAVHTRGLDFACD

SEQ ID NO: 9 - protein, 129 aa, synthetic construct
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 229 aa, synthetic construct
ESKYGPPCPSCPAPEFEGGPSVFLFPPKPKDTLMISRTPEVTCVVVDVSQEDPEVQFNWYVDGVEVHQAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 229 aa, synthetic construct
ESKYGPPCPPCPAPEFEGGPSVFLFPPKPKDTLMISRTPEVTCVVVDVSQEDPEVQFNWYVDGVEVHQAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 21 aa, synthetic construct
LCYLLDGILFIYGVILTALFL

SEQ ID NO: 14 - protein, 27 aa, synthetic construct
FWVLVVVGGVLACYSLLVTVAFIIFWV

SEQ ID NO: 15 - protein, 28 aa, synthetic construct
MFWVLVVVGGVLACYSLLVTVAFIIFWV

SEQ ID NO: 16 - protein, 22 aa, synthetic construct
MALIVLGGVAGLLLFIGLGIFF

SEQ ID NO: 17 - protein, 21 aa, synthetic construct
IYIWAPLAGTCGVLLLSLVIT

SEQ ID NO: 18 - protein, 23 aa, synthetic construct
IYIWAPLAGTCGVLLLSLVITLY

SEQ ID NO: 19 - protein, 24 aa, synthetic construct
IYIWAPLAGTCGVLLLSLVITLYC

SEQ ID NO: 20 - protein, 27 aa, synthetic construct
IISFFLALTSTALLFLLFFLTLRFSVV

SEQ ID NO: 21 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 41 aa, synthetic construct
RSKRSRLLHSDYMNMTPRRPGPTRKHYQPYAPPRDFAAYRS

SEQ ID NO: 23 - protein, 41 aa, synthetic construct
RSKRSRGGHSDYMNMTPRRPGPTRKHYQPYAPPRDFAAYRS

SEQ ID NO: 24 - protein, 42 aa, synthetic construct
KRGRKKLLYIFKQPFMRPVQTTQEEDGCSCRFPEEEEGGCEL

SEQ ID NO: 25 - protein, 42 aa, synthetic construct
ALYLLRRDQRLPPDAHKPPGGGSFRTPIQEEQADAHSTLAKI

SEQ ID NO: 26 - protein, 112 aa, synthetic construct
GPVPPSTAVRELIEELVNITQNQKAPLCNGSMVWSINLTAGMYCAALESLINVSGCSAIEKTQRMLSGFCPHKVSAGQFSSLHVRDTRIEVAQFVKDLLNHLKELFTEGQFN

SEQ ID NO: 27 - protein, 112 aa, synthetic construct
GPVPPSTAARELIEELFNITQNQKAPLCNGSMVWSINLTAGMYCAALESLINVSGCSAIEKTKRMLSGFCPHKVSAGQFPSLHVKKTRIEVAQFVKDLLIHLRKLFKEGQFN

SEQ ID NO: 28 - protein, 894 aa, synthetic construct
MLLLVTSLLLCELPHPAFLLIPGPVPPSTAVRELIEELVNITQNQKAPLCNGSMVWSINLTAGMYCAALESLINVSGCSAIEKTQRMLSGFCPHKVSAGQFSSLHVRDTRIEVAQFVKDLLNHLKELFTEGQFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 29 - protein, 525 aa, synthetic construct
GPVPPSTAVRELIEELVNITQNQKAPLCNGSMVWSINLTAGMYCAALESLINVSGCSAIEKTQRMLSGFCPHKVSAGQFSSLHVRDTRIEVAQFVKDLLNHLKELFTEGQFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 547 aa, synthetic construct
MLLLVTSLLLCELPHPAFLLIPGPVPPSTAVRELIEELVNITQNQKAPLCNGSMVWSINLTAGMYCAALESLINVSGCSAIEKTQRMLSGFCPHKVSAGQFSSLHVRDTRIEVAQFVKDLLNHLKELFTEGQFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 789 aa, synthetic construct
MLLLVTSLLLCELPHPAFLLIPGPVPPSTAVRELIEELVNITQNQKAPLCNGSMVWSINLTAGMYCAALESLINVSGCSAIEKTQRMLSGFCPHKVSAGQFSSLHVRDTRIEVAQFVKDLLNHLKELFTEGQFNESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 32 - protein, 420 aa, synthetic construct
GPVPPSTAVRELIEELVNITQNQKAPLCNGSMVWSINLTAGMYCAALESLINVSGCSAIEKTQRMLSGFCPHKVSAGQFSSLHVRDTRIEVAQFVKDLLNHLKELFTEGQFNESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 442 aa, synthetic construct
MLLLVTSLLLCELPHPAFLLIPGPVPPSTAVRELIEELVNITQNQKAPLCNGSMVWSINLTAGMYCAALESLINVSGCSAIEKTQRMLSGFCPHKVSAGQFSSLHVRDTRIEVAQFVKDLLNHLKELFTEGQFNESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 894 aa, synthetic construct
MLLLVTSLLLCELPHPAFLLIPGPVPPSTAARELIEELFNITQNQKAPLCNGSMVWSINLTAGMYCAALESLINVSGCSAIEKTKRMLSGFCPHKVSAGQFPSLHVKKTRIEVAQFVKDLLIHLRKLFKEGQFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 35 - protein, 525 aa, synthetic construct
GPVPPSTAARELIEELFNITQNQKAPLCNGSMVWSINLTAGMYCAALESLINVSGCSAIEKTKRMLSGFCPHKVSAGQFPSLHVKKTRIEVAQFVKDLLIHLRKLFKEGQFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 547 aa, synthetic construct
MLLLVTSLLLCELPHPAFLLIPGPVPPSTAARELIEELFNITQNQKAPLCNGSMVWSINLTAGMYCAALESLINVSGCSAIEKTKRMLSGFCPHKVSAGQFPSLHVKKTRIEVAQFVKDLLIHLRKLFKEGQFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 789 aa, synthetic construct
MLLLVTSLLLCELPHPAFLLIPGPVPPSTAARELIEELFNITQNQKAPLCNGSMVWSINLTAGMYCAALESLINVSGCSAIEKTKRMLSGFCPHKVSAGQFPSLHVKKTRIEVAQFVKDLLIHLRKLFKEGQFNESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 38 - protein, 420 aa, synthetic construct
GPVPPSTAARELIEELFNITQNQKAPLCNGSMVWSINLTAGMYCAALESLINVSGCSAIEKTKRMLSGFCPHKVSAGQFPSLHVKKTRIEVAQFVKDLLIHLRKLFKEGQFNESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 442 aa, synthetic construct
MLLLVTSLLLCELPHPAFLLIPGPVPPSTAARELIEELFNITQNQKAPLCNGSMVWSINLTAGMYCAALESLINVSGCSAIEKTKRMLSGFCPHKVSAGQFPSLHVKKTRIEVAQFVKDLLIHLRKLFKEGQFNESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 24 aa, synthetic construct
LEGGGEGRGSLLTCGDVEENPGPR

SEQ ID NO: 41 - protein, 354 aa, synthetic construct
LVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 42 - protein, 323 aa, synthetic construct
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

(19) United States
(12) Patent Application Publication
(10) Pub. No.: US 2026/0000764 A1
(43) Pub. Date: Jan. 1, 2026

(54) TARGETED CHIMERIC ANTIGEN RECEPTOR MODIFIED T CELLS FOR TREATMENT OF IL13Ralpha2 POSITIVE MALIGNANCIES

(71) Applicants: City of Hope, Duarte, CA (US); The Board of Trustees of the Leland Stanford Junior University, Stanford, CA (US)

(72) Inventors: Christine E. Brown, Duarte, CA (US); Lawrence Stern, Duarte, CA (US); Xin Yang, Duarte, CA (US); K. Christopher Garcia, Duarte, CA (US); Ignacio Moraga Gonzalez, Duarte, CA (US)

(21) Appl. No.: 19/313,530

(22) Filed: Aug. 28, 2025

Related U.S. Application Data
(63) Continuation of application No. 17/796,349, filed on Jul. 29, 2022, now Pat. No. 12,419,954.
(60) Provisional application No. 62/968,975, filed on Jan. 31, 2020.

(51) Int. Cl. A61K 40/31; A61K 40/11; A61K 40/42; A61P 35/00; C12N 15/86

(52) U.S. Cl. CPC A61K 40/31; A61K 40/11; A61K 40/4217; A61P 35/00; C12N 15/86; A61K 2239/31; A61K 2239/38; A61K 2239/47

(57) ABSTRACT

Chimeric antigen receptor molecules that include a variant IL-13. The variant IL-13 are more selective for IL13Rα2 than IL13Rα1 by virtue of weaker binding to IL13Rα1. The chimeric antigen receptors can be used to treat IL13Rα2 expressing cancers.

### CLAIM OF PRIORITY

This application is a continuation of U.S. patent application Ser. No. 17/796,349, filed on Jul. 29, 2022, which is a U.S. National Phase Application under 35 U.S.C. § 371 of International Application No. PCT/US2021/014008, filed on Jan. 19, 2021, which claims the benefit of U.S. Provisional Application No. 62/968,975, filed on Jan. 31, 2020. The entire contents of the foregoing are incorporated herein by reference.

### SEQUENCE LISTING

This application contains a Sequence Listing that has been submitted electronically as an XML file named “40056-0055002_SL_ST26.XML.” The XML file, created on Aug. 26, 2025, is 46,589 bytes in size. The material in the XML file is hereby incorporated by reference in its entirety.

### TECHNICAL FIELD

This disclosure concerns chimeric antigen receptors (CAR) engineered that bind to IL13 receptor, T cells expressing such CAR, methods of formulating such CAR T cells, and methods of use as anti-cancer agents.

### BACKGROUND

IL13Rα2 (Lupardus, Birnbaum et al. 2010), which is a versatile therapeutic target due to its rare expression in normal tissue (Debinski and Gibo 2000) and overexpression in many human cancers, including glioblastoma multiforme (GBM) (Thaci, Brown et al. 2014), pancreatic ductal adenocarcinoma (Shimamura, Fujisawa et al. 2010), melanoma (Beard, Abate-Daga et al. 2013), ovarian carcinoma (Kioi, Kawakami et al. 2006), clear cell renal cell carcinoma (Shibasaki, Yamasaki et al. 2015), breast cancer (Papageorgis, Ozturk et al. 2015), and lung cancer (Xie, Wu et al. 2015). A second IL13 receptor family member, IL13Rα1, interacts with IL13 with lower affinity (Lupardus, Birnbaum et al. 2010), and is ubiquitously expressed in healthy tissue (Debinski and Gibo 2000). Additionally, IL13Rα1 and IL4Rα, a receptor pair that binds IL13 with high affinity (Lupardus, Birnbaum et al. 2010) to mediate signaling through the JAK/STAT6 pathway (Murata, Taguchi et al. 1998), are co-expressed in pulmonary tissue (Hecker, Zaslona et al. 2010). Despite this wide expression of IL13 binding partners in healthy tissue, an IL13-ligand based CAR has shown safety in humans during clinical trials with locoregional central nervous system (CNS) delivery in GBM (Brown, Badie et al. 2015, Brown, Alizadeh et al. 2016), suggesting that toxicity from on-target/off-disease binding is not problematic in this context. However, for the treatment of systemic disease, the wide expression of IL13 binding partners outside of the diseased tissue could act as a sink for IL13-based therapy, resulting in safety concerns and possibly impeding trafficking to the disease site. Previous work in the field has attempted to address this problem by generating CARs derived from IL13 mutants containing mutations to direct binding away from IL13Rα1/IL4Rα. Mutations at E13 have yielded improved selectivity for IL13Rα2 over IL13Rα1 (Kahlon, Brown et al. 2004, Krebs, Chow et al. 2014), albeit with the E13Y mutation still allowing measurable recognition of IL13Rα1 in the context of both recombinant antigen and antigen-expressing cancer cells (Krebs, Chow et al. 2014). The addition of both E13K and R109K mutations into an IL13-based CAR also showed attenuated, but not abolished, recognition of IL13Rα1-expressing cancer cells relative to IL13Rα2-expressing cancer cells (Kong, Sengupta et al. 2012). While these examples are encouraging, additional mutations will be required to develop an IL13Rα2-specific IL13 mutant. Among the challenges in developing such molecules is that the impact of IL13 mutations on the function of an IL13 containing CAR cannot be predicted.

### SUMMARY

Described herein are IL13Rα2 targeted CAR that include a variant IL13 (“variant IL13 CAR”) to treat a variety of cancers.

The variant IL13 CAR described herein include a variant IL-13 comprising or consisting of the amino acid sequence:
GPVPPSTAVRELIEELVNITQNQKAPLCNGSMVWSINLTAGMYCAALESLINVSGCSA IEKTQRMLSGFCPHKVSAGQFSSLHVRDTRIEVAQFVKDLLNHLKELFTEGQFN (SEQ ID NO: 26); or comprising or consisting of the amino acid sequence:

(SEQ ID NO: 27)
GPVPPSTAARELIEELFNITQNQKAPLCNGSMVWSINLTAGMYCAALES
LINVSGCSAIEKTKRMLSGFCPHKVSAGQFPSLHVKKTRIEVAQFVKDL
LIHLRKLFKEGQFN.

Described herein is an IL13 CAR comprising a variant IL13 comprising the amino acid sequence of SEQ ID NO: 26 or 27, a spacer (e.g., comprising SEQ ID NO: 9, 10, 11 or 12), a transmembrane domain (e.g., comprising SEQ ID NO: 14, 15 or 22), a 41-BB co-stimulatory domain (comprising SEQ ID NO:24) and a CD3 zeta cytoplasmic domain (SEQ ID NO: 21).

Described herein is a nucleic acid molecule comprising a nucleotide sequence encoding a chimeric antigen receptor (CAR), wherein the chimeric antigen receptor comprises: a targeting domain comprising SEQ ID NO: 26 or SEQ ID NO: 27, a spacer, a transmembrane domain, a co-stimulatory domain, and a CD3ζ signaling domain. In various embodiments: the transmembrane domain is selected from: a CD4 transmembrane domain or variant thereof having 1-5 amino acid modifications, a CD8 transmembrane domain or variant thereof having 1-5 amino acid modifications, a CD28 transmembrane domain or a variant thereof having 1-5 amino acid modifications; the wherein the IL13 receptor targeting domain consists of the amino acid sequence of SEQ ID NO: 26 or SEQ ID NO: 27; the costimulatory domain is selected from: a 41BB costimulatory domain or variant thereof having 1-5 amino acid modifications, a CD28 costimulatory domain or variant thereof having 1-5 amino acid modifications; wherein the costimulatory domain is a 41BB costimulatory domain; the 41BB costimulatory domain comprises the amino acid sequence of SEQ ID NO: 24 or a variant thereof having 1-5 amino acid modifications; the CD3ζ signaling domain comprises the amino acid sequence of SEQ ID NO:21; a linker of 3 to 15 amino acids is located between the 4-1BB costimulatory domain and the CD3ζ signaling domain or variant thereof; the CAR comprises the amino acid sequence of SEQ ID NO: 28-39, or a variant thereof having 1-5amino acid modifications; the CAR comprises an amino acid sequence that is least about 80%, 85%, 90%, 95%, 96%, 97%, 98%, 99%, or 100% identical SEQ ID NO: 28; the CAR comprises an amino acid sequence that is least about 80%, 85%, 90%, 95%, 96%, 97%, 98%, 99%, or 100% identical SEQ ID NO: 29; the CAR comprises an amino acid sequence that is least about 80%, 85%, 90%, 95%, 96%, 97%, 98%, 99%, or 100% identical SEQ ID NO: 30;the CAR comprises an amino acid sequence that is least about 80%, 85%, 90%, 95%, 96%, 97%, 98%, 99%, or 100% identical SEQ ID NO: 31; the CAR comprises an amino acid sequence that is least about 80%, 85%, 90%, 95%, 96%, 97%, 98%, 99%, or 100% identical

SEQ ID NO: 32; the CAR comprises an amino acid sequence that is least about 80%, 85%, 90%, 95%, 96%, 97%, 98%, 99%, or 100% identical SEQ ID NO: 33; the CAR comprises an amino acid sequence that is least about 80%, 85%, 90%, 95%, 96%, 97%, 98%, 99%, or 100% identical SEQ ID NO: 34; the CAR comprises an amino acid sequence that is least about 80%, 85%, 90%, 95%, 96%, 97%, 98%, 99%, or 100% identical SEQ ID NO: 35; the CAR comprises an amino acid sequence that is least about 80%, 85%, 90%, 95%, 96%, 97%, 98%, 99%, or 100% identical SEQ ID NO: 36; the CAR comprises an amino acid sequence that is least about 80%, 85%, 90%, 95%, 96%, 97%, 98%, 99%, or 100% identical SEQ ID

NO: 37; the CAR comprises an amino acid sequence that is least about 80%, 85%, 90%, 95%, 96%, 97%, 98%, 99%, or 100% identical SEQ ID NO: 38; the CAR comprises an amino acid sequence that is least about 80%, 85%, 90%, 95%, 96%, 97%, 98%, 99%, or 100% identical SEQ ID NO:39. Also described is an expression vector comprising any of the forgoing nucleic acid molecules. Also described is a viral vector comprising any of the forgoing nucleic acid molecules.

Also described is a population of human T cells containing any of the forgoing nucleic acid molecules. Also described is a population of human T cells containing any of the forgoing expression vectors or viral vectors. In various embodiments, the population of human T cells comprise central memory T cells, naive memory T cells, pan T cells, or PBMC substantially depleted for CD25+ cells and CD14+ cells.

Also described is a method of treating a patient suffering from glioblastoma, pancreatic ductal adenocarcinoma, melanoma, ovarian carcinoma, renal cell carcinoma, breast cancer or lung cancer, comprising administering a population of autologous or allogeneic human T cells harboring a nucleic acid described herein. In various embodiments, the chimeric antigen receptor is administered locally or systemically; and the chimeric antigen receptor is administered by single or repeat dosing. In various embodiments, the chimeric antigen receptor substantially spares cells expressing IL13Rαl that do not express IL13Rα2.

Also described herein is a method of preparing CAR T cells comprising: providing a population of autologous or allogeneic human T cells and transducing the T cells by a vector comprising the nucleic acid molecule of claim 1.

Also described are T cells harboring a vector expressing the variant IL13 CAR. In various embodiments: at least 20%, 30%, or 40% of the transduced human T cells are central memory T cells; at least 30% of the transduced human T cells are CD4+ and CD62L+ or CD8+ and CD62L+. In various embodiments: the population of human T cells comprise a vector expressing a chimeric antigen receptor comprising an amino acid sequence selected from SEQ ID NOs: 27, 29, 30, and 31) or a variant thereof having 1-5 amino acid modifications (e.g., 1 or 2) amino acid modifications (e.g., substitutions); the population of human T cells comprises central memory T cells (T_{CM} cells) e.g., at least 20%, 30%, 40%, 50% 60%, 70%, 80% of the cells are T_{CM} cells, or the population of T cells comprises a combination of central memory T cells, naïve T cells and stem central memory cells (T_{CM/SCM/N} cells) e.g., at least 20%, 30%, 40%, 50% 60%, 70%, 80% of the cells are T_{CM/SCM/N} cells. In some embodiments, the population of T cells includes both CD4+ cells and CD8+ cells (e.g., at least 20% of the CD3+ T cells are CD4+ and at least 3% of the CD3+ T cells are CD8+ and at least 70, 80 or 90% are either CD4+ or CD8+; at least 15%, 20%, 25%, 30%, 35%, 40%, 50%, 60% of the cells CD3+ cells are CD4+ and at least 4%, 5%, 8%, 10%, 20 of the CD3+ cells are CD8+ cells). In some embodiments, the population of human T cells are autologous to the patient. In some embodiments, the population of human T cells are allogenic to the patient.

### IL13Rα2 Targeted CAR

The CAR described herein include a variant IL-13 comprising or consisting of the amino acid sequence:
GPVPPSTAVRELIEELVNITQNQKAPLCNGSMVWSINLTAGMYCAALESLINVSGCSA IEKTQRMLSGFCPHKVSAGQFSSLHVRDTRIEVAQFVKDLLNHLKELFTEGQFN (SEQ ID NO: 26); or comprising or consisting of the amino acid sequence GPVPPSTAARELIEELFNITQNQKAPLCNGSMVWSINLTAGMYCAALESLINVSGCSA IEKTKRMLSGFCPHKVSAGQFPSLHVKKTRIEVAQFVKDLLIHLRKLFKEGQFN (SEQ ID NO: 27) and the sequence.

A useful IL13 variant CAR can consist of or comprises the amino acid sequence of SEQ ID NO: 29, 32, 35 or 38 (mature CAR lacking a signal sequence) or the IL13 variant CAR can consist of or comprise the amino acid sequence of SEQ ID NO: 30, 33, 36 or 39 (immature CAR having a GMCSFRa signal sequence). Thus, thee CAR and can be expressed in a form that includes a signal sequence, e.g., a human GM-CSF receptor alpha signal sequence (MLLLVTSLLLCELPHPAFLLIP; SEQ ID NO:1). The CAR can be expressed with additional sequences that are useful for monitoring expression, for example, a T2A skip sequence and a truncated EGFRt. The CAR can be expressed with additional sequences that are useful for monitoring expression, for example, a T2A skip sequence and a truncated CD19t, e.g., SEQ ID NOs: 28, 31, 34 and 37. The variant IL13 CAR can comprise or consist of the amino acid sequence of any of SEQ ID NOs 28-29 with up to 1, 2, 3, 4 or 5 amino acid changes (preferably conservative amino acid changes).

In some embodiments, the nucleic acid encoding amino acid sequences SEQ ID NOs: 28-29 are codon optimized for expression in human cells.

### Spacer Region

The CAR described herein can include a spacer located between the variant IL13 domain (i.e., a variant IL13 comprising SEQ ID NO: xC4 or xD7) and the transmembrane domain. A variety of different spacers can be used. Some of them include at least portion of a human Fc region, for example a hinge portion of a human Fc region or a CH3 domain or variants thereof. Table 1 below provides various spacers that can be used in the CARs described

TABLE 1
Examples of Spacers

| Name | Length | Sequence |
|---|---|---|
| a3 | 3 aa | AAA |
| Iinker | 10 aa | GGGSSGGGSG (SEQ ID NO: 2) |
| IgG4 hinge (S→P) | 12 aa | ESKYGPPCPPCP (SEQ ID NO: 3) |
| (S228P) | | |
| IgG4 hinge | 12 aa | ESKYGPPCPSCP (SEQ ID NO: 4) |
| IgG4 hinge (S228P) + linker | 22 aa | ESKYGPPCPPCPGGGSSGGGSG (SEQ ID NO: 5) |
| CD28 hinge | 39 aa | IEVMYPPPYLDNEKSNGTIIHVKGKHLCPSPLFPGPSKP (SEQ ID NO: 6) |
| CD8 hinge-48 aa | 48 aa | AKPTTTPAPRPPTPAPTIASQPLSLRPEACRPAAGGAVHTRGLDFACD (SEQ |
| | | ID NO: 7) |
| CD8 hinge-45 aa | 45 aa | TTTPAPRPPTPAPTIASQPLSLRPEACRPAAGGAVHTRGLDFACD (SEQ ID |
| | | NO: 8) |
| IgG4(HL-CH3) | 129 aa | ESKYGPPCPPCPGGGSSGGGSGGQPREPQVYTLPPSQEEMTKNQVSLTCL |
| (includes S228P in hinge) | | VKGFYPSDIAVEWESNGQPENNYKTTPPVLDSDGSFFLYSRLTVDKSRWQE |
| | | GNVFSCSVMHEALHNHYTQKSLSLSLGK (SEQ ID NO: 9) |
| IgG4(L235E, N297Q) | 229 aa | JESKYGPPCPSCPAPEFEGGPSVFLFPPKPKDTLMISRTPEVTCVVVDVSQED |
| | | PEVQFNWYVDGVEVHQAKTKPREEQFQSTYRVVSVLTVLHQDWLNGKEY |
| | | KCKVSNKGLPSSIEKTISKAKGQPREPQVYTLPPSQEEMTKNQVSLTCLVKG |
| | | FYPSDIAVEWESNGQPENNYKTTPPVLDSDGSFFLYSRLTVDKSRWQEGN |
| | | VFSCSVMHEALHNHYTQKSLSLSLGK (SEQ ID NO: 10) |
| IgG4(S228P, L235E, N297Q) | 229 aa | JESKYGPPCPPCPAPEFEGGPSVFLFPPKPKDTLMISRTPEVTCVVVDVSQED |
| | | PEVQFNWYVDGVEVHQAKTKPREEQFQSTYRVVSVLTVLHQDWLNGKEY |
| | | KCKVSNKGLPSSIEKTISKAKGQPREPQVYTLPPSQEEMTKNQVSLTCLVKG |
| | | FYPSDIAVEWESNGQPENNYKTTPPVLDSDGSFFLYSRLTVDKSRWQEGN |
| | | VFSCSVMHEALHNHYTQKSLSLSLGK (SEQ ID NO: 11) |
| IgG4(CH3) | 107 aa | GQPREPQVYTLPPSQEEMTKNQVSLTCLVKGFYPSDIAVEWESNGQPENN |
| | | YKTTPPVLDSDGSFFLYSRLTVDKSRWQEGNVFSCSVMHEALHNHYTQKSL |
| | | SLSLGK (SEQ ID NO: 12) |

Some spacer regions include all or part of an immunoglobulin (e.g., IgG1, IgG2, IgG3, IgG4) hinge region, i.e., the sequence that falls between the CH1 and CH2 domains of an immunoglobulin, e.g., an IgG4 Fc hinge or a CD8 hinge. Some spacer regions include an immunoglobulin CH3 domain or both a CH3 domain and a CH2 domain. The immunoglobulin derived sequences can include one or more amino acid modifications, for example, 1, 2, 3, 4 or 5 substitutions, e.g., substitutions that reduce off-target binding.

The hinge/linker region can also comprise a IgG4 hinge region having the sequence ESKYGPPCPSCP (SEQ ID NO:4) or ESKYGPPCPPCP (SEQ ID NO:3). The hinge/linger region can also comprise the sequence ESKYGPPCPPCP (SEQ ID NO:3) followed by the linker sequence GGGSSGGGSG (SEQ ID NO:2) followed by IgG4 CH3 sequence GQPREPQVYTLPPSQEEMTKNQVSLTCLVKGFYPSDIAVEWESNGQPENNYKTTPPV LDSDGSFFLYSRLTVDKSRWQEGNVFSCSVMHEALHNHYTQKSLSLSLGK (SEQ ID NO: 12). Thus, the entire linker/spacer region can comprise the sequence: ESKYGPPCPPCPGGGSSGGGSGGQPREPQVYTLPPSQEEMTKNQVSLTCLVKGFYPS DIAVEWESNGQPENNYKTTPPVLDSDGSFFLYSRLTVDKSRWQEGNVFSCSVMHEA LHNHYTQKSLSLSLGK (SEQ ID NO:11). In some cases, the spacer has 1, 2, 3, 4, or 5 single amino acid changes (e.g., conservative changes) compared to SEQ ID NO: 10 or 11. In some cases, the IgG4 Fc hinge/linker region is mutated at two positions (L235E; N297Q) in a manner that reduces binding by Fc receptors (FcRs) (e.g., comprises or consists of SEQ ID NO: 10 or 11).

### Transmembrane Domain

A variety of transmembrane domains can be used in the. Table 2 includes examples of suitable transmembrane domains. Where a spacer region is present, the transmembrane domain (TM) is located carboxy terminal to the spacer region.

TABLE 2
Examples of Transmembrane Domains

| Name | Accession | Length | Sequence |
|---|---|---|---|
| CD3z | J04132.1 | 21 aa | LCYLLDGILFIYGVILTALFL (SEQ ID NO: 13) |
| CD28 | NM_006139 | 27 aa | FWVLVVVGGVLACYSLLVTVAFIIFWV (SEQ ID NO: 14) |
| CD28(M) | NM_006139 | 28 aa | MFWVLVVVGGVLACYSLLVTVAFIIFWV (SEQ ID NO: 15) |
| CD4 | M35160 | 22 aa | MALIVLGGVAGLLLFIGLGIFF (SEQ ID NO: 16) |
| CD8tm | NM_001768 | 21 aa | IYIWAPLAGTCGVLLLSLVIT (SEQ ID NO: 17) |
| CD8tm2 | NM_001768 | 23 aa | IYIWAPLAGTCGVLLLSLVITLY (SEQ ID NO: 18) |
| CD8tm3 | NM_001768 | 24 aa | IYIWAPLAGTCGVLLLSLVITLYC (SEQ ID NO: 19) |
| 41BB | NM_001561 | 27 aa | ISFFLALTSTALLFLLFF LTLRFSVV (SEQ ID NO: 20) |

### Costimulatory Domain

The costimulatory domain can be any domain that is suitable for use with a CD3ζ signaling domain. In some cases the co-signaling domain is a 4-1BB co-signaling domain that includes a sequence that is at least 90%, at least 95%, at least 98% identical to or identical to: KRGRKKLLYIFKQPFMRPVQTTQEEDGCSCRFPEEEEGGCEL (SEQ ID NO:24). In some cases, the 4-1BB co-signaling domain has 1, 2, 3, 4 of 5 amino acid changes (preferably conservative) compared to SEQ ID NO:24.

The costimulatory domain(s) are located between the transmembrane domain and the CD3ζ signaling domain. Table 3 includes examples of suitable costimulatory domains together with the sequence of the CD3ζ signaling domain.

TABLE 3
CD32 Domain and Examples of Costimulatory Domains

| Name | Accession | Length | Sequence |
|---|---|---|---|
| CD37 | J04132.1 | 113 aa | RVKFSRSADAPAYQQGQNQLYNELNLGRREEYDVLDKRRGR |
| | | | DPEMGGKPRRKNPQEGLYNELQKDKMAEAYSEIGMKGERR |
| | | | RGKGHDGLYQGLSTATKDTYDALHMQALPPR (SEQ ID |
| | | | NO: 21) |
| CD28 | NM_006139 | 42 aa | RSKRSRLLHSDYMNMTPRRPGPTRKHYQPYAPPRDFAAYRS |
| | | | (SEQ ID NO: 22) |
| CD28gg* | NM 006139 | 42 aa | RSKRSRGGHSDYMNMTPRRPGPTRKHYQPYAPPRDFAAYR |
| | | | S (SEQ ID NO: 23) |
| 41BB | NM_001561 | 42 aa | KRGRKKLLYIFKQPFMRPVQTTQEEDGCSCRFPEEEEGGCEL |
| | | | (SEQ ID NO: 24) |
| OX40 | | 42 aa | ALYLLRRDQRLPPDAHKPPGGGSFRTPIQEEQADAHSTLAKI |
| | | | (SEQ ID NO: 25) |

In various embodiments: the costimulatory domain is selected from the group consisting of: a costimulatory domain depicted in Table 3 or a variant thereof having 1-5 (e.g., 1 or 2) amino acid modifications, a CD28 costimulatory domain or a variant thereof having 1-5 (e.g., 1 or 2) amino acid modifications, a 4-1BB costimulatory domain or a variant thereof having 1-5 (e.g., 1 or 2) amino acid modifications and an OX40 costimulatory domain or a variant thereof having 1-5 (e.g., 1 or 2) amino acid modifications. In certain embodiments, a 4-1BB costimulatory domain or a variant thereof having 1-5 (e.g., 1 or 2) amino acid modifications in present. In some embodiments there are two costimulatory domains, for example a CD28 co-stimulatory domain or a variant thereof having 1-5 (e.g., 1 or 2) amino acid modifications (e.g., substitutions) and a 4-1BB co-stimulatory domain or a variant thereof having 1-5 (e.g., 1 or 2) amino acid modifications (e.g., substitutions). In various embodiments the 1-5 (e.g., 1 or 2) amino acid modification are substitutions. The costimulatory domain is amino terminal to the CD3ζ signaling domain and a short linker consisting of 2-10, e.g., 3 amino acids (e.g., GGG) is can be positioned between the costimulatory domain and the CD3ζ signaling domain.

### CD3ζ Signaling Domain

The CD3ζ Signaling domain can be any domain that is suitable for use with a CD3ζ signaling domain. In some cases, the CD3ζ signaling domain includes a sequence that is at least 90%, at least 95%, at least 98% identical to or identical to:
RVKFSRSADAPAYQQGQNQLYNELNLGRREEYDVLDKRRGRDPEMGGKPRRKNPQ EGLYNELQKDKMAEAYSEIGMKGERRRGKGHDGLYQGLSTATKDTYDALHMQAL PPR (SEQ ID NO:21). In some cases, the CD3ζ signaling has 1, 2, 3, 4 of 5 amino acid changes (preferably conservative) compared to SEQ ID NO:21.

### Truncated EGFR and Truncated CD19

The CD3ζ signaling domain can be followed by a ribosomal skip sequence (e.g., LEGGGEGRGSLLTCGDVEENPGPR; SEQ ID NO:40) and a truncated EGFR having a sequence that is at least 90%, at least 95%, at least 98% identical to or identical to: LVTSLLLCELPHPAFLLIPRKVCNGIGIGEFKDSLSINATNIKHFKNCTSISGDLHILPVA FRGDSFTHTPPLDPQELDILKTVKEITGFLLIQAWPENRTDLHAFENLEIIRGRTKQHG QFSLAVVSLNITSLGLRSLKEISDGDVIISGNKNLCYANTINWKKLFGTSGQKTKIISN RGENSCKATGQVCHALCSPEGCWGPEPRDCVSCRNVSRGRECVDKCNLLEGEPREF VENSECIQCHPECLPQAMNITCTGRGPDNCIQCAHYIDGPHCVKTCPAGVMGENNTL VWKYADAGHVCHLCHPNCTYGCTGPGLEGCPTNGPKIPSIATGMVGALLLLLVVAL GIGLFM (SEQ ID NO: 41). In some cases, the truncated EGFR has 1, 2, 3, 4 of 5 amino acid changes (preferably conservative) compared to SEQ ID NO: 41.

Alternatively the CD3ζ signaling domain can be followed by a ribosomal skip sequence (e.g., LEGGGEGRGSLLTCGDVEENPGPR; SEQ ID NO:40) and a truncated CD19R having a sequence that is at least 90%, at least 95%, at least 98% identical to or identical to:

(SEQ ID NO: 42)
MPPPRLLFFLLFLTPMEVRPEEPLVVKVEEGDNAVLQCLKGTSDGPTQQ
LTWSRESPLKPFLKLSLGLPGLGIHMRPLAIWLFIFNVSQQMGGFYLCQ
PGPPSEKAWQPGWTVNVEGSGELFRWNVSDLGGLGCGLKNRSSEGPSSP
SGKLMSPKLYVWAKDRPEIWEGEPPCVPPRDSLNQSLSQDLTMAPGSTL
WLSCGVPPDSVSRGPLSWTHVHPKGPKSLLSLELKDDRPARDMWVMETG
LLLPRATAQDAGKYYCHRGNLTMSFHLEITARPVLWHWLLRTGGWKVSA
VTLAYLIFCLCSLVGILHLQRALVLRRKR

An amino acid modification refers to an amino acid substitution, insertion, and/or deletion in a protein or peptide sequence. An “amino acid substitution” or “substitution” refers to replacement of an amino acid at a particular position in a parent peptide or protein sequence with another amino acid. A substitution can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. The following are examples of various groupings of amino acids: 1) Amino acids with nonpolar R groups: Alanine, Valine, Leucine, Isoleucine, Proline, Phenylalanine, Tryptophan, Methionine; 2) Amino acids with uncharged polar R groups: Glycine, Serine, Threonine, Cysteine, Tyrosine, Asparagine, Glutamine; 3) Amino acids with charged polar R groups (negatively charged at pH 6.0): Aspartic acid, Glutamic acid; 4) Basic amino acids (positively charged at pH 6.0): Lysine, Arginine, Histidine (at pH 6.0). Another grouping may be those amino acids with phenyl groups: Phenylalanine, Tryptophan, and Tyrosine.

In some cases, the CAR can be produced using a vector in which the CAR open reading frame is followed by a T2A ribosome skip sequence and a truncated EGFR (EGFRt) or truncated CD19. (CD19t) In this arrangement, co-expression of EGFRt or CD19t provides an inert, non-immunogenic surface marker that allows for accurate measurement of gene modified cells, and enables positive selection of gene-modified cells, as well as efficient cell tracking of the therapeutic T cells in vivo following adoptive transfer. Efficiently controlling proliferation to avoid cytokine storm and off-target toxicity is an important hurdle for the success of T cell immunotherapy. The EGFRt or CD19t incorporated in the lentiviral vector can act as suicide gene to ablate the CAR+ T cells in cases of treatment-related toxicity.

The CAR described herein can be produced by any means known in the art, though preferably it is produced using recombinant DNA techniques. Nucleic acids encoding the several regions of the chimeric receptor can be prepared and assembled into a complete coding sequence by standard techniques of molecular cloning known in the art (genomic library screening, overlapping PCR, primer-assisted ligation, site-directed mutagenesis, etc.) as is convenient. The resulting coding region is preferably inserted into an expression vector and used to transform a suitable expression host cell line, preferably a T lymphocyte, and most preferably an autologous T lymphocyte.

Various T cell subsets isolated from the patient can be transduced with a vector for CAR expression. Central memory T cells are one useful T cell subset. Central memory T cell can be isolated from peripheral blood mononuclear cells (PBMC) by selecting for CD45RO+/CD62L+ cells, using, for example, the CliniMACS® device to immunomagnetically select cells expressing the desired receptors. The cells enriched for central memory T cells can be activated with anti-CD3/CD28, transduced with, for example, a lentiviral vector that directs the expression of the CAR as well as a non-immunogenic surface marker for in vivo detection, ablation, and potential ex vivo selection. The activated/genetically modified CAR T T cells can be expanded in vitro with IL-2/IL-15 and then cryopreserved. Additional methods of preparing CAR T cells can be found in PCT/US2016/043392.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety for any and all purposes. In case of conflict, the present specification, including definitions, will control.

Other features and advantages of the invention will be apparent from the following detailed description and figures, and from the claims.

### DESCRIPTION OF DRAWINGS

FIG. 1A-1C: Binding profile of IL13 D7 and IL13 C4 for IL13 receptors. (A) Structural superposition of the IL13/IL13Rα1 and IL13/IL13Rα2 complexes. The docking mode of IL13 with IL13Rα1 is essentially identical to its docking mode with IL13Rα2. Right inset boxes show amino acids on IL13 forming the IL13Rα1-IL13Rα2-site II and site III binding interfaces (yellow: IL13 binding IL13Rα1; green: IL13 binding IL13Rα2). IL13 uses the same amino acids to form the IL13Rα1-and IL13Rα2-site II and site III binding interface. (B) IL13 wild type binds with high efficiency IL13Rα1 and IL13Rα2. IL13 C4 and D7 specifically bind IL13Rα2. (C) IL13 variants binding parameters for IL13Rα1 and IL13Rα2 measured by surface plasmon resonance.

FIG. 2A-2F: IL-13Rα1 and IL-13Rα2 binding affinities of the IL-13 variant. (A-C) Surface plasmon resonance measurements of IL-13, IL-13 D7 and IL-13 C4 for IL-13Rα1. IL-13Rα1 was immobilized on the surface of the chip and the indicated doses of IL-13 or the two IL-13 mutants were flowed. (D-F) Surface plasmon resonance measurements of IL-13,IL-13 D7 and IL-13 C4 for IL-13Rα2. IL-13Rα2 was immobilized on the surface of the chip and the indicated doses of IL-13 or the two IL-13 mutants were flowed.

FIG. 3A-3C: Signal activation and activities potencies exhibited by the IL13 D7 and IL13 C4 muteins. (A) Levels of pSTAT6, measured by flow cytometry, induced by IL13 or the two IL13 mutants in A549 cells stimulated with the indicated doses for 15 min. Data are presented as mean±range of two replicates. (B) Levels of CCL26 gene induction induced by IL13 and the two mutants in the human fibroblast KA23 measured by qPCR. Data are presented as mean±range of two replicates. (C) Levels of TF-1 cell proliferation exhibited by IL13 and the two IL13 muteins after 96 hr of stimulation with the indicated doses. Data are presented as mean±range of two replica.

FIG. 4A-4C: IL13-variant CARs. (A) Schematic of IL13-CAR constructs containing wild-type (WT), E12Y, C4 or D7 binding domains. CARs also include an IgG4 (EQ)-Fc linker, a CD28 transmembrane and cytoplasmic signaling domain, and CD3ζ. Constructs also contain a truncated CD19 (CD19t) separated from the CAR by a ribosomal T2A skip sequence. (B) Histograms showing anti-CD19 for cell transduction (left panel) and anti-Fc staining for CAR expression (right panel) for untransduced (Mock), IL13 WT, E12Y, C4 and D7 CARs by flow cytometry. Percent positive cells (and MFI) are denoted in each histogram. One representative experiment is shown. (C) Dose response binding affinity curves for the IL13 WT, E12Y and C4 and D7 CAR T cells to human IL 13Rα2-Fc (left) or IL13Rα1-Fc (right). Data are presented as mean±standard deviation of three independent trials.

FIG. 5A-5C: Detection of IL-13Rα2, IL-13Rα1 and IL-4R on cancer cell lines. (A) Flow cytometry histograms showing either IL-13Rα2, IL-13Rα1 or IL-4R expression in the following adherent human cancer cell lines: (1) PBT030-2, (2) U251T, (3) A549, (4) HT1080P, (5) HT1080-IL13Rα1, (6) HT1080-IL13Rα1/IL4R, and (7) HT1080-IL13Rα2. The cancer cell lines were enzymatically dissociated into single cells and immediately stained with antigen markers (IL13Rα2, IL13Rα1, IL4R) at 4C° in the dark for 30 mins. Cells were then assessed by flow and the antigen expressions of viable tumors were displayed as histograms. Values located within each histogram represent the % expression with the mean fluorescence intensity (MFI) in parentheses. (B) Western blot showing the protein expression of IL13Rα2 and IL13Rα1, with B-actin as a loading control. (C) qPCR analysis showing the mRNA expression of IL13Rα2, IL13Rα1, IL4R and codon optimized IL13Rα2. For both the western blot and qPCR, protein or mRNA lysates were isolated, respectively, from the previously mentioned cancer cell lines. All data shown are means +/−sem.

FIG. 6A-6F: IL13 WT, E12Y, C4, and D7 CARs mediate comparable IL13Rα2-dependent in vitro effector function. IL13Rα2-dependent degranulation (A) and IFN-γ production (B) for IL13 WT, E12Y, C4, and D7 CAR and untransduced (Mock) T cells. T cell lines were co-cultured at a 1:1 effector to target (E:T) ratio with IL13Rα2+glioblastoma lines U251T and PBT030-2 or HT1080 engineered to express IL13Rα2 (HT1080-IL13Rα2); after 5 hrs the degranulation marker CD107a and intracellular IFN-γ was measured by flow cytometry and percent positive cells graphed (mean±SD; n=3 wells). (C) Secreted IFN-γ production by indicated T cell lines in response to increasing concentrations of immobilized recombinant human IL13Rα2-Fc (mean±SD; n=3 wells). p<0.0001 for all CAR T cell IL13-variants and mock T cells. (D) Tumor killing by IL13-CAR and mock T cell lines cultured at a 1:10 E:T ratio for 2 days. Flow cytometry was used to determine the remaining viable tumor count and tumor killing was determined as a % normalized to mock (mean±SD; n=3 wells). (E) CAR T cell killing in experiments using four separate donors at 1:50 E:T against PBT030-2, U251T and HT1080IL-13Rα2 showing similar levels of tumor killing for WT, E12Y, C4, and D7. Plots represent remaining tumor cells at day 7 (Dotted line represents seeded tumor cells). (F) Live CAR T cells in experiments presented following 7-day co-culture presented in (E). No live CAR T cells were observed in Tumor only and Mock CAR T cell experiments; levels of CAR T cell persistence were as follows: WT>D7>E12Y>C4 (Dotted line represents seeded CAR T cells). Panels of A-F are representative of at least three independent experiments. All data shown from A-F are means +/−sem. (**** p<0.0001, *** p<0.005, ** p<0.01, * p<0.05).

FIG. 7A-7C: IL13-E12Y, -C4 and -D7 mutein CARs exhibit similar levels of therapeutic efficacy in vivo against a human glioma xenografts. In vivo efficacy is similar across WT and mutein CARs against a human glioma xenografts. (A) NSG mice stereotactically implanted into the right forebrain with 0.1×105 EGFP-ffLuc+PBT030-2 tumorspheres. On day 8 mice received either no treatment, 0.3×10⁶ injection of Mock Tn/mem (no CAR) T cells or a 0.3×106 injection of the following CAR positive Tn/mem cells: IL13 WT, IL13ζ28 (E12Y), IL13ζ28 (C4), IL13ζ28 (D7). (n=6-8) [Two of the mice in group D7 died early during treatment.] Representative flux images are shown on day 150 post engraftment. (B) Kaplan-Meier survival curves (summary data for three experiments) demonstrate comparable survival at day 150 (dotted line represents day 9+/−1 day) for mice treated with the IL13-CAR variants, with IL13-WT, -E13Y, -C4 and -D7 CAR T cells all improving survival as compared to Mock T cells (p≤0.0001 Mantle-Cox log rank test (n=14-18). (C) Bar graph summarizing data from three separate experiments indicating the number of tumor free, tumor relapse and euthanized untreated mice, and mice treated with the IL13-CAR variants, with IL13-WT, -E13Y, -C4 and -D7 CAR T cells at day 150 post tumor implantation

FIG. 8A-8F: IL13-E12Y, -C4 and -D7 mutein CARs exhibit similar levels of therapeutic efficacy in vivo against a human glioma model. (A-C) Quantification of ffLuc+ flux (photons/sec show C4 and D7 IL-13 mutein CAR T cells have comparable potency to the WT and E12Y single mutant IL13-CARs. Dotted lines represent the flux values of individual mice while the colored solid line represent the average flux of that treatment cohort. Data are represent three independent experiments (n=6-8). (D-F) Kaplan-Meier survival curves for the corresponding adjacent flux values of the individual demonstrating comparable survival and tumor relapse rate for mice treated with the IL13-CAR variants, with IL13-WT, -E13Y, -C4 and -D7 CAR T cells all improving survival as compared to Mock T cell treated and untreated mice (p≤0.0001 Mantle-Cox log rank test (n=6-8).

FIG. 9A-9F: IL13 C4 and D7 mutein CAR T Cells showed reduced recognition of IL13Rα1. IL13Rα1-depedendent degranulation (A) and IFN-γ production (B) for IL13 WT, E12Y, C4 and D7 CAR T cells co-cultured with IL13Rα1+A549, HT1080 parental, and HT1080 engineered to express either IL13Rα1 or both IL13Rα1 and IL4R as described in FIG. 7. (C) Secreted IFN-γ production by indicated T cell lines in response to increasing concentrations of immobilized recombinant human IL13Rα1-Fc (mean±SD; n=3 wells). (D) IL13Rα1-dependent cell killing of the indicated CAR and target lines at a 1:10 E:T ratio for 2 days. (A)-(D) are representative of at least three independent experiments. (E) Winn assay evaluating A549 (0.1×10⁶) tumor engraftment after co-culture with CAR and mock T cell lines (E:T ratio 10:1; 2 hr pre-incubation and engraftment on day 0; n=4). (F) NSG mice xenotransplated with 0.5×10⁶ HT1080 IL13Rα1/IL4R in 1:1 ratio of media to matrigel, followed by treatment with either no T Cells, or 5×10⁶ mock T cells or the various mutein CAR T cells on day 4. All data shown on A-F are means +/−sem. (**** p<0.0001, *** p<0.005, ** p<0.01, * p<0.05).

FIG. 10A-10B: Sequence of a CAR having C4 mutein targeting domain. Amino acid sequence of (A) IL13 (C4)-IgG4 (L235E,N297Q)-CD28gg-Zeta (CO)-T2A-CD19t, including the signal sequence and T2A-CD19t marker (SEQ ID NO: 28); and (B) same CAR without signal sequence and T2A-CD19t marker (SEQ ID NO: 29). In both cases, the various domains are indicated. The CAR sequence is in bold and the marker sequences is not. SEQ NO: 30 includes the signal sequence, but not the T2A-CD19t marker.

FIG. 11A-11B: Sequence of a CAR having C4 mutein targeting domain. Amino acid sequence of (A) IL13 (C4)-IgG4 (HL-CH3)-CD4tm-41BB-Zeta (CO)-T2A-CD19t (BspEI), including the signal sequence and T2A-CD19t (BspEI) marker (SEQ ID NO: 31); and (B) same CAR without signal sequence and T2A-CD19t BspEI) marker (SEQ ID NO: 32). In both cases, the various domains are indicated. The CAR sequence is in bold and the marker sequences is not. SEQ NO: 33 includes the signal sequence, but not the T2A-CD19t marker.

FIG. 12A-12B: Sequence of a CAR having D7 mutein targeting domain. Amino acid sequence of (A) IL13 (D7)-IgG4 (L235E,N297Q)-CD28gg-Zeta (CO)-T2A-CD19t, including the signal sequence and T2A-CD19t marker (SEQ ID NO: 34); and (B) same CAR without signal sequence and T2A-CD19t marker (SEQ ID NO: 35). In both cases, the various domains are indicated. The CAR sequence is in bold and the marker sequences is not. SEQ NO: 36 includes the signal sequence, but not the T2A-CD19t marker.

FIG. 13A-13B: Sequence of a CAR having D7 mutein targeting domain. Amino acid sequence of (A) IL13 (D7)-IgG4 (HL-CH3)-CD4tm-41BB-Zeta (CO)-T2A-CD19t (BspEI), including the signal sequence and T2A-CD19t (BspEI) marker (SEQ ID NO: 37); and (B) same CAR without signal sequence and T2A-CD19t (BspEI) marker (SEQ ID NO: 38). In both cases, the various domains are indicated. The CAR sequence is in bold and the marker sequences is not. SEQ NO: 39 includes the signal sequence, but not the T2A-CD19t marker.

FIGS. 14: D7 and C4 mutein CAR traffic away from lung. CAR were administered to mice and presence in the lungs was assessed by luminescence on Day 8

### DETAILED DESCRIPTION

In this disclosure the generation and anti-tumor efficacy of CAR with a variant IL13 domain targeting IL13Rα2 are described. The CAR T cells exhibited potent antigen-dependent cytotoxicity against L13Rα2-expressing human cancer lines. In vivo delivery of the CAR T cells in a murine tumor model conferred elimination of antigen-positive disease and extension of overall survival.

### IL13Rα2 Targeted CAR

The CAR described herein include a variant IL-13 comprising or consisting of the amino acid sequence:
GPVPPSTAVRELIEELVNITQNQKAPLCNGSMVWSINLTAGMYCAALESLINVSGCSA IEKTQRMLSGFCPHKVSAGQFSSLHVRDTRIEVAQFVKDLLNHLKELFTEGQFN (SEQ ID NO: 26); or comprising or consisting of the amino acid sequence GPVPPSTAARELIEELFNITQNQKAPLCNGSMVWSINLTAGMYCAALESLINVSGCSA IEKTKRMLSGFCPHKVSAGQFPSLHVKKTRIEVAQFVKDLLIHLRKLFKEGQFN (SEQ ID NO: 27) and the sequence.

A useful IL13 variant CAR can consist of or comprises the amino acid sequence of SEQ ID NO: 29, 32, 35 or 38 (mature CAR lacking a signal sequence) or the IL13 variant CAR can consist of or comprise the amino acid sequence of SEQ ID NO: 30, 33, 36 or 39 (immature CAR having a GMCSFRa signal sequence). Thus, thee CAR and can be expressed in a form that includes a signal sequence, e.g., a human GM-CSF receptor alpha signal sequence (MLLLVTSLLLCELPHPAFLLIP; SEQ ID NO:1). The CAR can be expressed with additional sequences that are useful for monitoring expression, for example, a T2A skip sequence and a truncated EGFRt. The CAR can be expressed with additional sequences that are useful for monitoring expression, for example, a T2A skip sequence and a truncated CD19t, e.g., SEQ ID NOs: 28, 31, 34 and 37. The variant IL13 CAR can comprise or consist of the amino acid sequence of any of SEQ ID NOs 28-29 with up to 1, 2, 3, 4 or 5 amino acid changes (preferably conservative amino acid changes).

In some embodiments, the nucleic acid encoding amino acid sequences SEQ ID NOs: 28-29 are codon optimized for expression in human cells.

### Spacer Region

The CAR described herein can include a spacer located between the variant IL13 domain (i.e., a variant IL13 comprising SEQ ID NO: xC4 or xD7) and the transmembrane domain. A variety of different spacers can be used. Some of them include at least portion of a human Fc region, for example a hinge portion of a human Fc region or a CH3 domain or variants thereof. Table 1 below provides various spacers that can be used in the CARs described

TABLE 1
Examples of Spacers

| Name | Length | Sequence |
|---|---|---|
| a3 | 3 aa | AAA |
| Iinker | 10 aa | GGGSSGGGSG (SEQ ID NO: 2) |
| IgG4 hinge (S→P) | 12 aa | ESKYGPPCPPCP (SEQ ID NO: 3) |
| (S228P) | | |
| IgG4 hinge | 12 aa | ESKYGPPCPSCP (SEQ ID NO: 4) |
| IgG4 hinge (S228P) + linker | 22 aa | ESKYGPPCPPCPGGGSSGGGSG (SEQ ID NO: 5) |
| CD28 hinge | 39 aa | IEVMYPPPYLDNEKSNGTIIHVKGKHLCPSPLFPGPSKP (SEQ ID NO: 6) |
| CD8 hinge-48 aa | 48 aa | AKPTTTPAPRPPTPAPTIASQPLSLRPEACRPAAGGAVHTRGLDFACD (SEQ |
| | | ID NO: 7) |
| CD8 hinge-45 aa | 45 aa | TTTPAPRPPTPAPTIASQPLSLRPEACRPAAGGAVHTRGLDFACD (SEQ ID |
| | | NO: 8) |
| IgG4(HL-CH3) | 129 aa | ESKYGPPCPPCPGGGSSGGGSGGQPREPQVYTLPPSQEEMTKNQVSLTCL |
| (includes S228P in hinge) | | VKGFYPSDIAVEWESNGQPENNYKTTPPVLDSDGSFFLYSRLTVDKSRWQE |
| | | GNVFSCSVMHEALHNHYTQKSLSLSLGK (SEQ ID NO: 9) |
| IgG4(L235E, N297Q) | 229 aa | JESKYGPPCPSCPAPEFEGGPSVFLFPPKPKDTLMISRTPEVTCVVVDVSQED |
| | | PEVQFNWYVDGVEVHQAKTKPREEQFQSTYRVVSVLTVLHQDWLNGKEY |
| | | KCKVSNKGLPSSIEKTISKAKGQPREPQVYTLPPSQEEMTKNQVSLTCLVKG |
| | | FYPSDIAVEWESNGQPENNYKTTPPVLDSDGSFFLYSRLTVDKSRWQEGN |
| | | VFSCSVMHEALHNHYTQKSLSLSLGK (SEQ ID NO: 10) |
| IgG4(S228P, L235E, N297Q) | 229 aa | JESKYGPPCPPCPAPEFEGGPSVFLFPPKPKDTLMISRTPEVTCVVVDVSQED |
| | | PEVQFNWYVDGVEVHQAKTKPREEQFQSTYRVVSVLTVLHQDWLNGKEY |
| | | KCKVSNKGLPSSIEKTISKAKGQPREPQVYTLPPSQEEMTKNQVSLTCLVKG |
| | | FYPSDIAVEWESNGQPENNYKTTPPVLDSDGSFFLYSRLTVDKSRWQEGN |
| | | VFSCSVMHEALHNHYTQKSLSLSLGK (SEQ ID NO: 11) |
| IgG4(CH3) | 107 aa | GQPREPQVYTLPPSQEEMTKNQVSLTCLVKGFYPSDIAVEWESNGQPENN |
| | | YKTTPPVLDSDGSFFLYSRLTVDKSRWQEGNVFSCSVMHEALHNHYTQKSL |
| | | SLSLGK (SEQ ID NO: 12) |

Some spacer regions include all or part of an immunoglobulin (e.g., IgG1, IgG2, IgG3, IgG4) hinge region, i.e., the sequence that falls between the CH1 and CH2 domains of an immunoglobulin, e.g., an IgG4 Fc hinge or a CD8 hinge. Some spacer regions include an immunoglobulin CH3 domain (called CH3 or ACH2) or both a CH3 domain and a CH2 domain. The immunoglobulin derived sequences can include one or more amino acid modifications, for example, 1, 2, 3, 4 or 5 substitutions, e.g., substitutions that reduce off-target binding.

The hinge/linker region can also comprise an IgG4 hinge region having the sequence ESKYGPPCPSCP (SEQ ID NO:4) or ESKYGPPCPPCP (SEQ ID NO:3). The hinge/linger region can also comprise the sequence ESKYGPPCPPCP (SEQ ID NO:3) followed by the linker sequence GGGSSGGGSG (SEQ ID NO:2) followed by IgG4 CH3 sequence GQPREPQVYTLPPSQEEMTKNQVSLTCLVKGFYPSDIAVEWESNGQPENNYKTTPPV LDSDGSFFLYSRLTVDKSRWQEGNVFSCSVMHEALHNHYTQKSLSLSLGK (SEQ ID NO: 12). Thus, the entire linker/spacer region can comprise the sequence: ESKYGPPCPPCPGGGSSGGGSGGQPREPQVYTLPPSQEEMTKNQVSLTCLVKGFYPS DIAVEWESNGQPENNYKTTPPVLDSDGSFFLYSRLTVDKSRWQEGNVFSCSVMHEA LHNHYTQKSLSLSLGK (SEQ ID NO:11). In some cases, the spacer has 1, 2, 3, 4, or 5 single amino acid changes (e.g., conservative changes) compared to SEQ ID NO:11. In some cases, the IgG4 Fc hinge/linker region that is mutated at two positions (L235E; N297Q) in a manner that reduces binding by Fc receptors (FcRs).

### Transmembrane Domain

A variety of transmembrane domains can be used in the. Table 2 includes examples of suitable transmembrane domains. Where a spacer region is present, the transmembrane domain (TM) is located carboxy terminal to the spacer region.

TABLE 2
Examples of Transmembrane Domains

| Name | Accession | Length | Sequence |
|---|---|---|---|
| CD3z | J04132.1 | 21 aa | LCYLLDGILFIYGVILTALFL (SEQ ID NO: 13) |
| CD28 | NM_006139 | 27 aa | FWVLVVVGGVLACYSLLVTVAFIIFWV (SEQ ID NO: 14) |
| CD28(M) | NM_006139 | 28 aa | MFWVLVVVGGVLACYSLLVTVAFIIFWV (SEQ ID NO: 15) |
| CD4 | M35160 | 22 aa | MALIVLGGVAGLLLFIGLGIFF (SEQ ID NO: 16) |
| CD8tm | NM_001768 | 21 aa | IYIWAPLAGTCGVLLLSLVIT (SEQ ID NO: 17) |
| CD8tm2 | NM_001768 | 23 aa | IYIWAPLAGTCGVLLLSLVITLY (SEQ ID NO: 18) |
| CD8tm3 | NM_001768 | 24 aa | IYIWAPLAGTCGVLLLSLVITLYC (SEQ ID NO: 19) |
| 41BB | NM_001561 | 27 aa | ISFFLALTSTALLFLLFF LTLRFSVV (SEQ ID NO: 20) |

### Costimulatory Domain

The costimulatory domain can be any domain that is suitable for use with a CD3ζ signaling domain. In some cases the co-signaling domain is a 4-1BB co-signaling domain that includes a sequence that is at least 90%, at least 95%, at least 98% identical to or identical to: KRGRKKLLYIFKQPFMRPVQTTQEEDGCSCRFPEEEEGGCEL (SEQ ID NO:24). In some cases, the 4-1BB co-signaling domain has 1, 2, 3, 4 of 5 amino acid changes (preferably conservative) compared to SEQ ID NO:24.

The costimulatory domain(s) are located between the transmembrane domain and the CD3ζ signaling domain. Table 3 includes examples of suitable costimulatory domains together with the sequence of the CD3ζ signaling domain.

TABLE 3
CD32 Domain and Examples of Costimulatory Domains

| Name | Accession | Length | Sequence |
|---|---|---|---|
| CD37 | J04132.1 | 113 aa | RVKFSRSADAPAYQQGQNQLYNELNLGRREEYDVLDKRRGR |
| | | | DPEMGGKPRRKNPQEGLYNELQKDKMAEAYSEIGMKGERR |
| | | | RGKGHDGLYQGLSTATKDTYDALHMQALPPR (SEQ ID |
| | | | NO: 21) |
| CD28 | NM_006139 | 42 aa | RSKRSRLLHSDYMNMTPRRPGPTRKHYQPYAPPRDFAAYRS |
| | | | (SEQ ID NO: 22) |
| CD28gg* | NM 006139 | 42 aa | RSKRSRGGHSDYMNMTPRRPGPTRKHYQPYAPPRDFAAYR |
| | | | S (SEQ ID NO: 23) |
| 41BB | NM_001561 | 42 aa | KRGRKKLLYIFKQPFMRPVQTTQEEDGCSCRFPEEEEGGCEL |
| | | | (SEQ ID NO: 24) |
| OX40 | | 42 aa | ALYLLRRDQRLPPDAHKPPGGGSFRTPIQEEQADAHSTLAKI |
| | | | (SEQ ID NO: 25) |

In various embodiments: the costimulatory domain is selected from the group consisting of: a costimulatory domain depicted in Table 3 or a variant thereof having 1-5 (e.g., 1 or 2) amino acid modifications, a CD28 costimulatory domain or a variant thereof having 1-5 (e.g., 1 or 2) amino acid modifications, a 4-1BB costimulatory domain or a variant thereof having 1-5 (e.g., 1 or 2) amino acid modifications and an OX40 costimulatory domain or a variant thereof having 1-5 (e.g., 1 or 2) amino acid modifications. In certain embodiments, a 4-1BB costimulatory domain or a variant thereof having 1-5 (e.g., 1 or 2) amino acid modifications in present. In some embodiments there are two costimulatory domains, for example a CD28 co-stimulatory domain or a variant thereof having 1-5 (e.g., 1 or 2) amino acid modifications (e.g., substitutions) and a 4-1BB co-stimulatory domain or a variant thereof having 1-5 (e.g., 1 or 2) amino acid modifications (e.g., substitutions). In various embodiments the 1-5 (e.g., 1 or 2) amino acid modification are substitutions. The costimulatory domain is amino terminal to the CD3ζ signaling domain and a short linker consisting of 2-10, e.g., 3 amino acids (e.g., GGG) is can be positioned between the costimulatory domain and the CD3ζ signaling domain.

### CD3ζ Signaling Domain

The CD3ζ Signaling domain can be any domain that is suitable for use with a CD3ζ signaling domain. In some cases, the CD3ζ signaling domain includes a sequence that is at least 90%, at least 95%, at least 98% identical to or identical to:
RVKFSRSADAPAYQQGQNQLYNELNLGRREEYDVLDKRRGRDPEMGGKPRRKNPQ EGLYNELQKDKMAEAYSEIGMKGERRRGKGHDGLYQGLSTATKDTYDALHMQAL PPR (SEQ ID NO:21). In some cases, the CD3ζ signaling has 1, 2, 3, 4 of 5 amino acid changes (preferably conservative) compared to SEQ ID NO:21.

### Truncated EGFR and Truncated CD19

The CD3ζ signaling domain can be followed by a ribosomal skip sequence (e.g., LEGGGEGRGSLLTCGDVEENPGPR; SEQ ID NO:40) and a truncated EGFR having a sequence that is at least 90%, at least 95%, at least 98% identical to or identical to:
LVTSLLLCELPHPAFLLIPRKVCNGIGIGEFKDSLSINATNIKHFKNCTSISGDLHILPVA FRGDSFTHTPPLDPQELDILKTVKEITGFLLIQAWPENRTDLHAFENLEIIRGRTKQHG QFSLAVVSLNITSLGLRSLKEISDGDVIISGNKNLCYANTINWKKLFGTSGQKTKIISN RGENSCKATGQVCHALCSPEGCWGPEPRDCVSCRNVSRGRECVDKCNLLEGEPREF VENSECIQCHPECLPQAMNITCTGRGPDNCIQCAHYIDGPHCVKTCPAGVMGENNTL VWKYADAGHVCHLCHPNCTYGCTGPGLEGCPTNGPKIPSIATGMVGALLLLLVVAL GIGLFM (SEQ ID NO: 41). In some cases, the truncated EGFR has 1, 2, 3, 4 of 5 amino acid changes (preferably conservative) compared to SEQ ID NO: 41.

Alternatively the CD3ζ signaling domain can be followed by a ribosomal skip sequence (e.g., LEGGGEGRGSLLTCGDVEENPGPR; SEQ ID NO:40) and a truncated CD19R having a sequence that is at least 90%, at least 95%, at least 98% identical to or identical to:

(SEQ ID NO: 42)
MPPPRLLFFLLFLTPMEVRPEEPLVVKVEEGDNAVLQCLKGTSDGPTQQ
LTWSRESPLKPFLKLSLGLPGLGIHMRPLAIWLFIFNVSQQMGGFYLCQ
PGPPSEKAWQPGWTVNVEGSGELFRWNVSDLGGLGCGLKNRSSEGPSSP
SGKLMSPKLYVWAKDRPEIWEGEPPCVPPRDSLNQSLSQDLTMAPGSTL
WLSCGVPPDSVSRGPLSWTHVHPKGPKSLLSLELKDDRPARDMWVMETG
LLLPRATAQDAGKYYCHRGNLTMSFHLEITARPVLWHWLLRTGGWKVSA
VTLAYLIFCLCSLVGILHLQRALVLRRKR

An amino acid modification refers to an amino acid substitution, insertion, and/or deletion in a protein or peptide sequence. An “amino acid substitution” or “substitution” refers to replacement of an amino acid at a particular position in a parent peptide or protein sequence with another amino acid. A substitution can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. The following are examples of various groupings of amino acids: 1) Amino acids with nonpolar R groups: Alanine, Valine, Leucine, Isoleucine, Proline, Phenylalanine, Tryptophan, Methionine; 2) Amino acids with uncharged polar R groups: Glycine, Serine, Threonine, Cysteine, Tyrosine, Asparagine, Glutamine; 3) Amino acids with charged polar R groups (negatively charged at pH 6.0): Aspartic acid, Glutamic acid; 4) Basic amino acids (positively charged at pH 6.0): Lysine, Arginine, Histidine (at pH 6.0). Another grouping may be those amino acids with phenyl groups: Phenylalanine, Tryptophan, and Tyrosine.

In some cases, the CAR can be produced using a vector in which the CAR open reading frame is followed by a T2A ribosome skip sequence and a truncated EGFR (EGFRt) or truncated CD19. (CD19t) In this arrangement, co-expression of EGFRt or CD19t provides an inert, non-immunogenic surface marker that allows for accurate measurement of gene modified cells, and enables positive selection of gene-modified cells, as well as efficient cell tracking of the therapeutic T cells in vivo following adoptive transfer. Efficiently controlling proliferation to avoid cytokine storm and off-target toxicity is an important hurdle for the success of T cell immunotherapy. The EGFRt or CD19t incorporated in the lentiviral vector can act as suicide gene to ablate the CAR+ T cells in cases of treatment-related toxicity.

The CAR described herein can be produced by any means known in the art, though preferably it is produced using recombinant DNA techniques. Nucleic acids encoding the several regions of the chimeric receptor can be prepared and assembled into a complete coding sequence by standard techniques of molecular cloning known in the art (genomic library screening, overlapping PCR, primer-assisted ligation, site-directed mutagenesis, etc.) as is convenient. The resulting coding region is preferably inserted into an expression vector and used to transform a suitable expression host cell line, preferably a T lymphocyte, and most preferably an autologous T lymphocyte.

Various T cell subsets isolated from the patient can be transduced with a vector for CAR expression. Central memory T cells are one useful T cell subset. Central memory T cell can be isolated from peripheral blood mononuclear cells (PBMC) by selecting for CD45RO+/CD62L+ cells, using, for example, the CliniMACS® device to immunomagnetically select cells expressing the desired receptors. The cells enriched for central memory T cells can be activated with anti-CD3/CD28, transduced with, for example, a lentiviral vector that directs the expression of the CAR as well as a non-immunogenic surface marker for in vivo detection, ablation, and potential ex vivo selection. The activated/genetically modified CAR T T cells can be expanded in vitro with IL-2/IL-15 and then cryopreserved. Additional methods of preparing CAR T cells can be found in PCT/US2016/043392.

### EXAMPLES

The invention is further described in the following examples, which do not limit the scope of the invention described in the claims.

### Example 1

IL13 variants exhibit selective binding to IL13Rα2

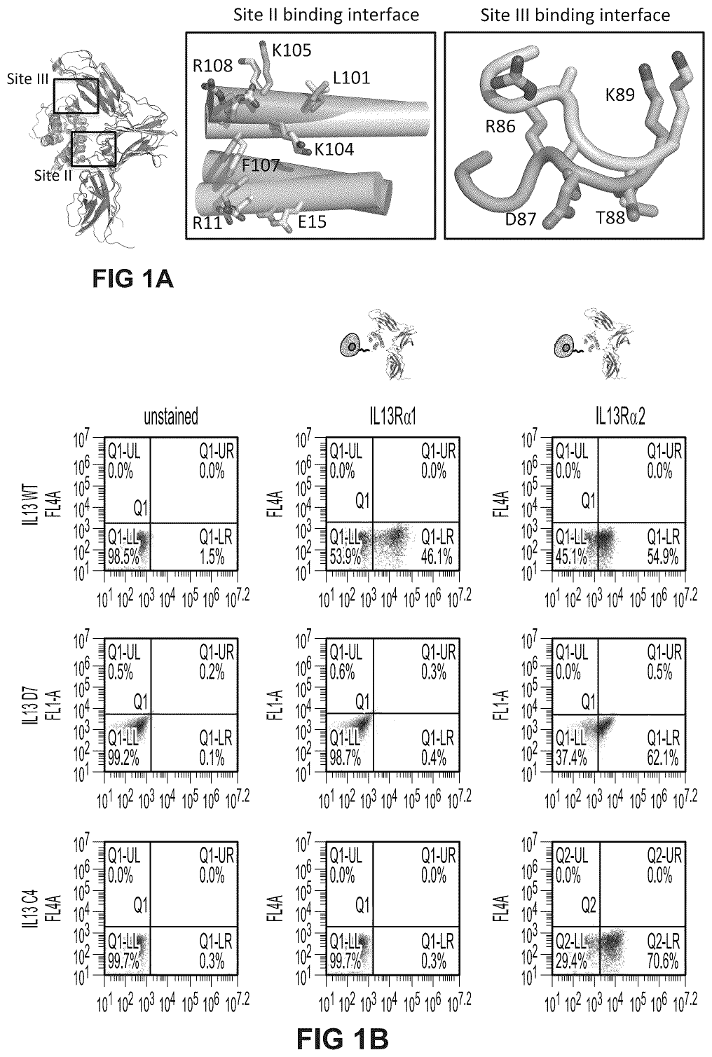

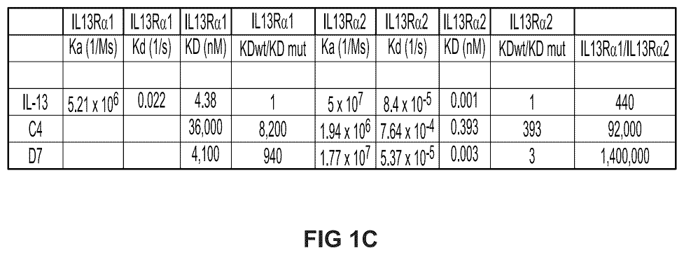

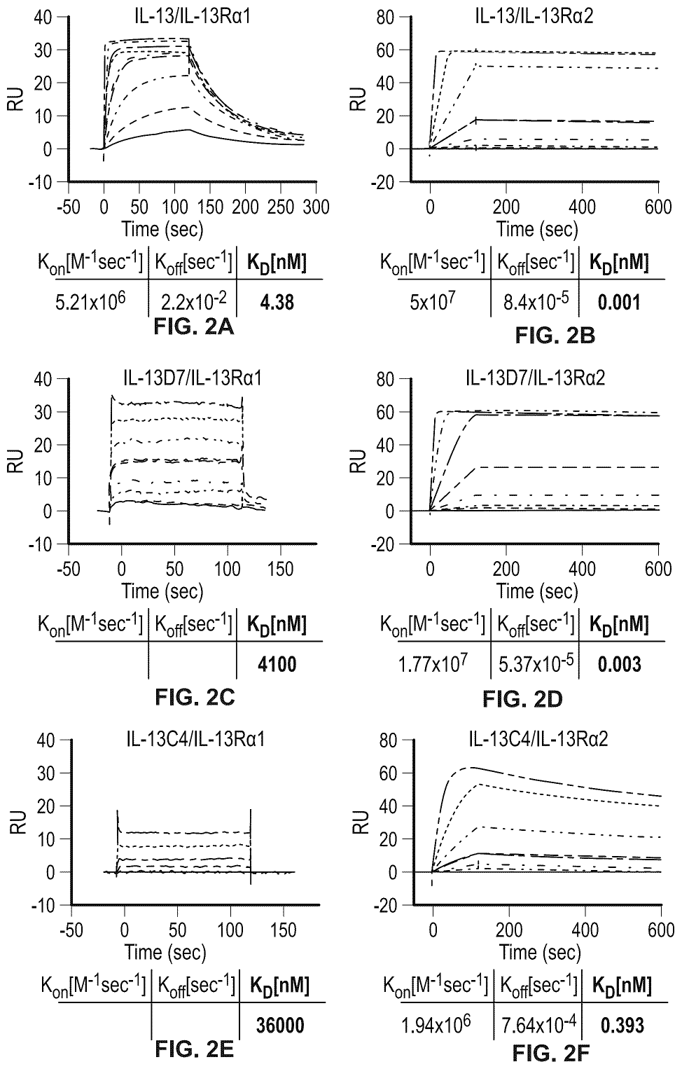

IL13 C4 and IL13 D7 are variants that have reduced affinity for IL13Rα1 relative to wild-type IL13 (WT) (Moraga, Richter et al. 2015). Because IL13Rα1 and IL13Rα2 share very similar binding interfaces on IL13 (FIG. 1A), IL13 C4 and IL13 D7 could exhibit a similar affinity decrease towards IL13Rα2. To assess receptor binding, WT, C4, and D7 were each displayed on the surface of yeast. Yeast were incubated with either recombinant IL13Rα1 or IL13Rα2 and binding was analyzed by flow cytometry (FIG. 1B). As expected, WT bound strongly to both IL13Rα1 and IL13Rα2. Both C4 and D7 showed IL13Rα2 binding comparably to WT, but no detectable binding to IL13Rα1 at the concentration tested. Surface plasmon resonance studies further confirmed these observations (FIG. 1C and FIG. 2A-F). WT, C4, and D7 all exhibit subnanomolar affinity for IL13Rα2 (0.001 nM, 0.393 nM, and 0.003 nM, respectively). However, the three variants strongly differ in binding affinity to IL13Rα1, confirming previous reporting (Moraga, Richter et al. 2015). These differences result in a several-log range of selectivity for IL13Rα2 relative to IL13Rα1, with WT, C4, and D7 exhibiting 440-fold, 92,000-fold, and 1,400,000-fold stronger affinity for IL13Rα2 relative to IL13Rα1.

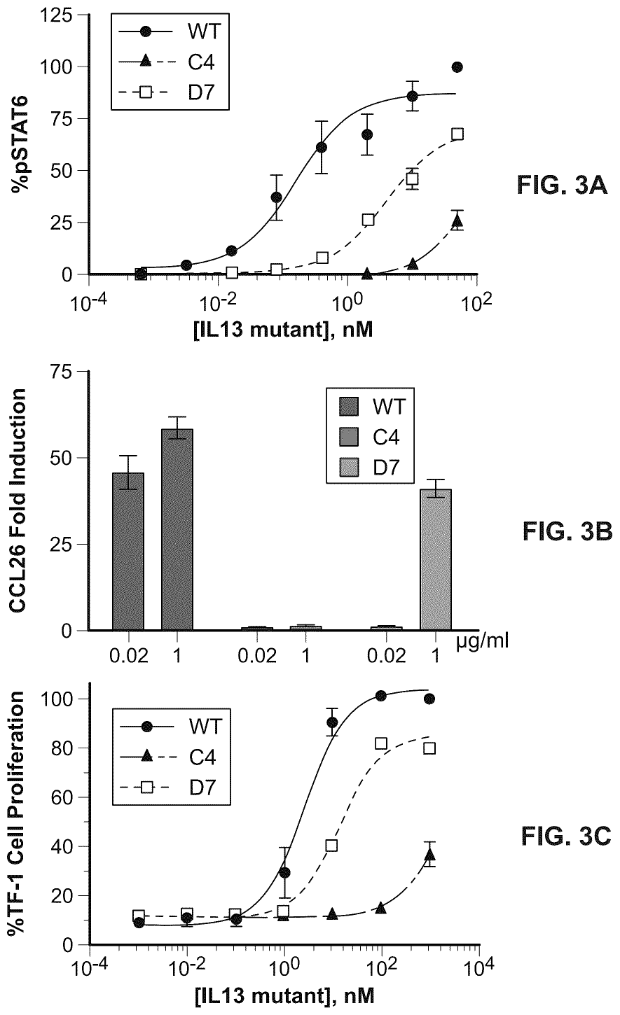

To further confirm this selectivity, IL 13 WT, IL13 C4, and IL13 D7 were tested in a variety of biological assays wherein activation through IL13Rα1 would yield functional outputs. First, soluble IL13 variants were titrated against IL13-responsive A549 cells for 15 minutes, and the phosphorylation of STAT6 was assessed by flow cytometry (FIG. 3A). Relative to WT, D7 and C4 both showed attenuated STAT6 phosphorylation, with the latter being more pronounced. Second, human fibroblasts were stimulated with IL13 variants for 8 hours, and upregulation of the CCL26 gene was assessed by qPCR (FIG. 3B). At both concentrations tested, WT induced strong upregulation of CCL26. D7 yielded an attenuated response, only inducing upregulation at the higher concentration of 1 μg/mL. C4 yielded essentially no response at both concentrations tested. Finally, IL13 variants were titrated against TF-1 for 96 hours, and cell proliferation was tracked (FIG. 3C). Relative to WT, treatment with D7 and C4 yielded attenuated proliferation, with the latter again being more pronounced.

### Example 2

### Variant IL13 CAR

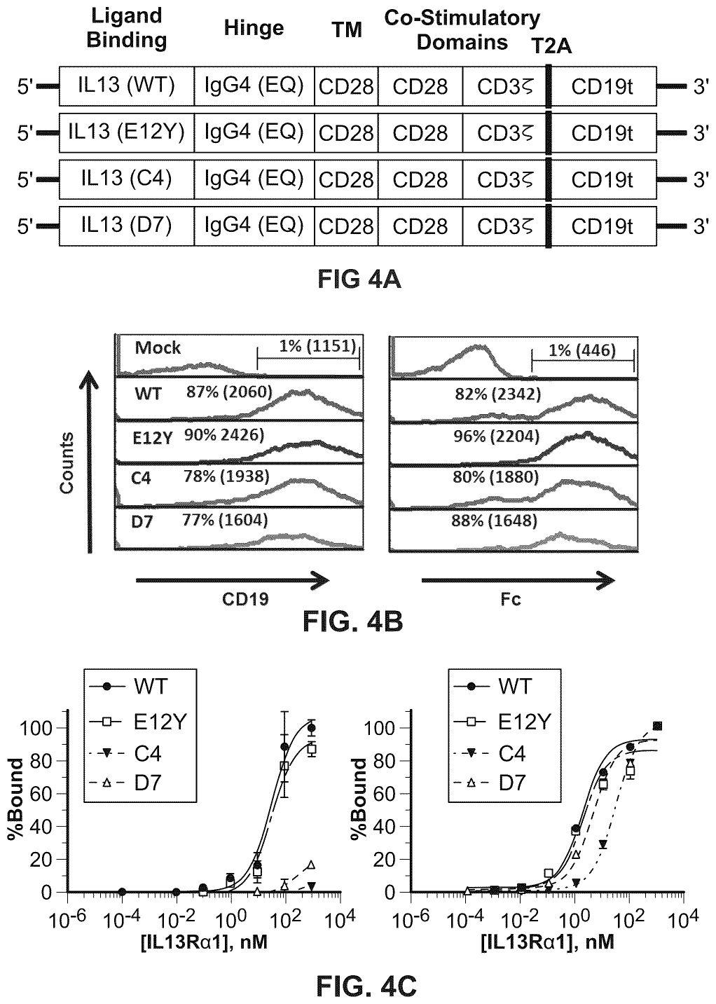

CAR that include C4 IL13, D7 IL13, E12Y IL13 or WT IL13 were generated. The four CAR were otherwise identical and included the CD28 co-stimulatory. The CAR included: a IgG4-Fc linker mutated at two sites within the CH2 region (L235E; N297Q) to reduce Fc receptor binding, a CD28 transmembrane domain, and the intracellular signaling domain of CD28 in series with CD3ζ (FIG. 4A). Our group and others have observed that IL13-based CARs with CD28-based costimulation (vs 4-1BB costimulation) have more sensitive recognition of IL13Rα1 expression (Brombacher, Nono et al. 2017). Therefore, in order to better differentiate IL13Rα2 specificity, we used CD28 costimulation as part of the CAR backbone. In addition, CAR cassettes have a T2A ribosome skip sequence followed by a truncated CD19 used as a marker of lentiviral transduction efficiency, cell tracking, and enrichment. Flow cytometry analysis of CD19t for cell transduction and IgG4-Fc for CAR detection confirm comparable expression for the four IL13-CAR variants (FIG. 4B). For all in vitro and in vivo studies, IL13-CARs were engineered in naïve/memory T cells (Tn/mem). Tn/mem are enriched CD62L+ naïve and memory T cells, including both the central memory (Tcm) and stem cell memory (Tscm) populations, along with naïve T cells (Tn).

To assess target specificity of the IL13-CAR T cells, we evaluated their binding affinities to recombinant human IL13Rα2 and IL-13Rα1 in dose-response curves (FIG. 4C). T cells stably expressing the indicated IL13-based constructs were titrated with biotinylated IL13Rα1 or IL13Rα2 for 1 hr at 4° C. The level of receptor bound to IL13 was measured by incubating the cells with streptavidin coupled to Alexa Fluor-647. E12Y and D7 CAR T cells bind to IL13Rα2 with similar affinity to WT, i.e., 1.8 nM (95% CI: 1.0-3.7 nM), 4.2 nM (95% CI: 2.8-6.3 nM) and 1.7 nM (95% CI: 1.2-2.4 nM) respectively, whereas C4 displayed a weaker affinity, 29 nM (95% CI: 23-36) (FIG. 4C, right panel). Notably, the affinities of IL13-derived CARs for IL13Rα2 are two to three orders of magnitude weaker than those of the free proteins. By contrast, D7 and C4 CAR T cells exhibited minimal binding to IL13Rα1 on the assayed concentration range, whereas WT and E12Y had nearly identical affinity (34 nM; 95% CI: 21-54 nM for both variants) (FIG. 4C, left panel).

### Example 3

### Functional Characterization of C4 and D7 IL13 Variant CAR T Cells in IL13Rα2 Targeting

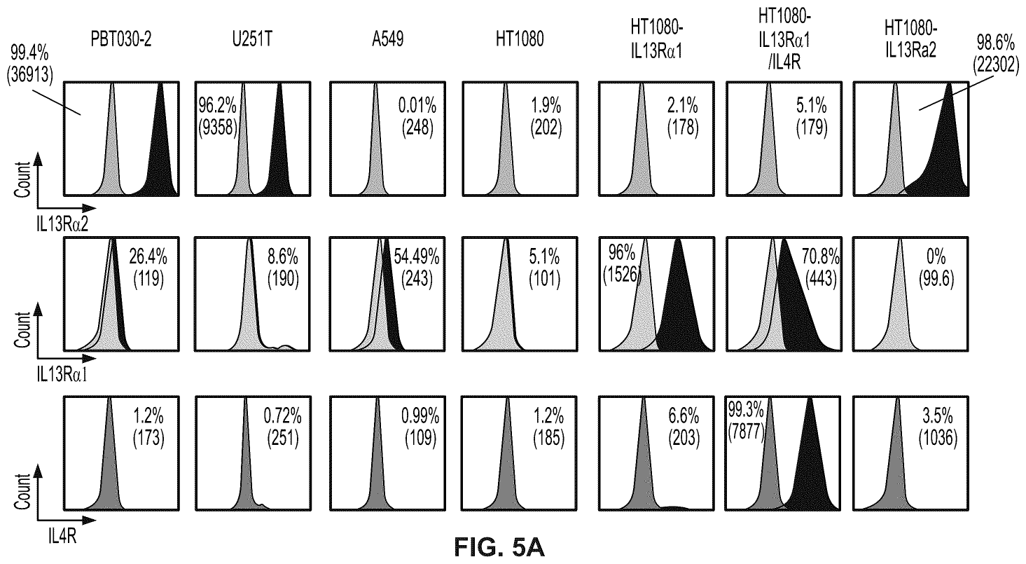

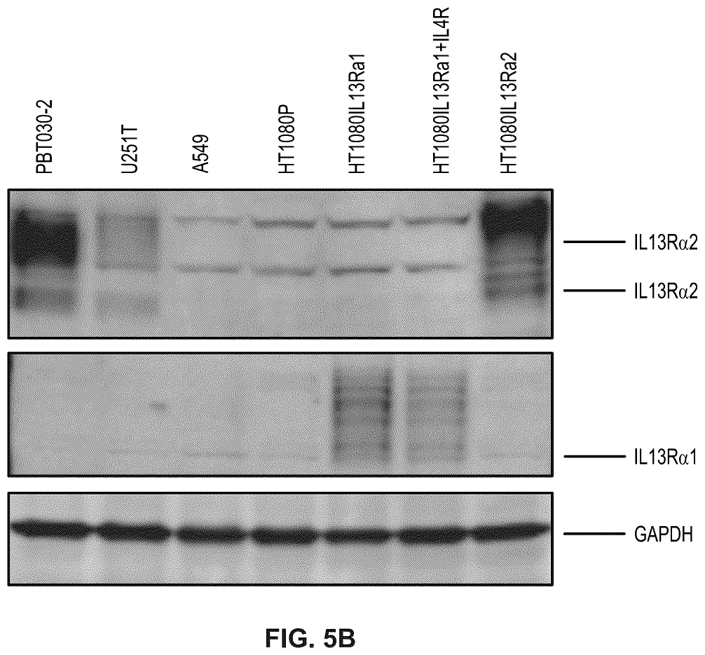

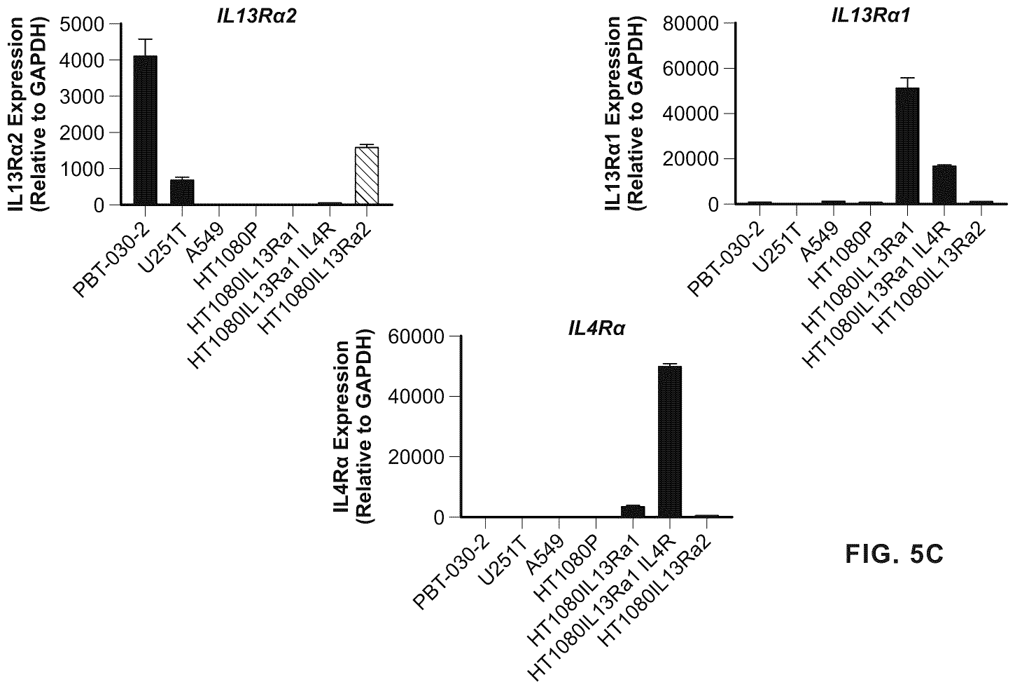

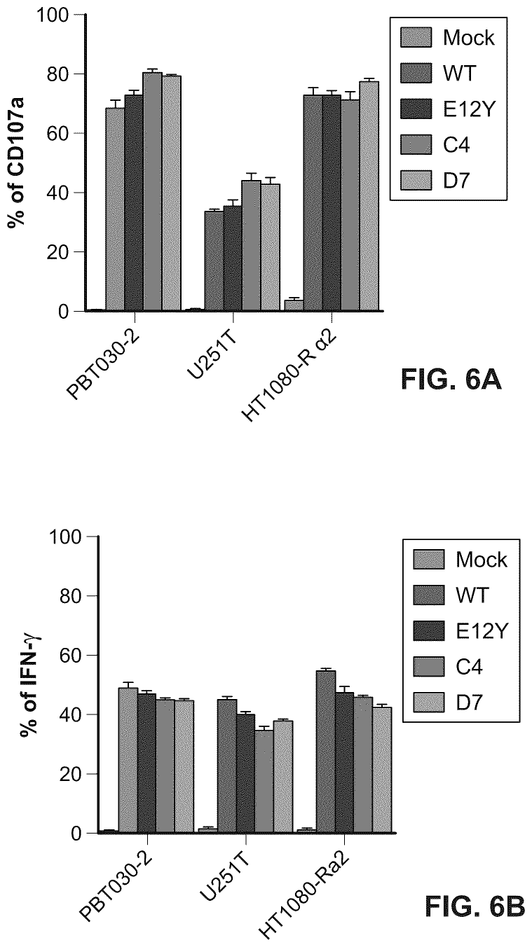

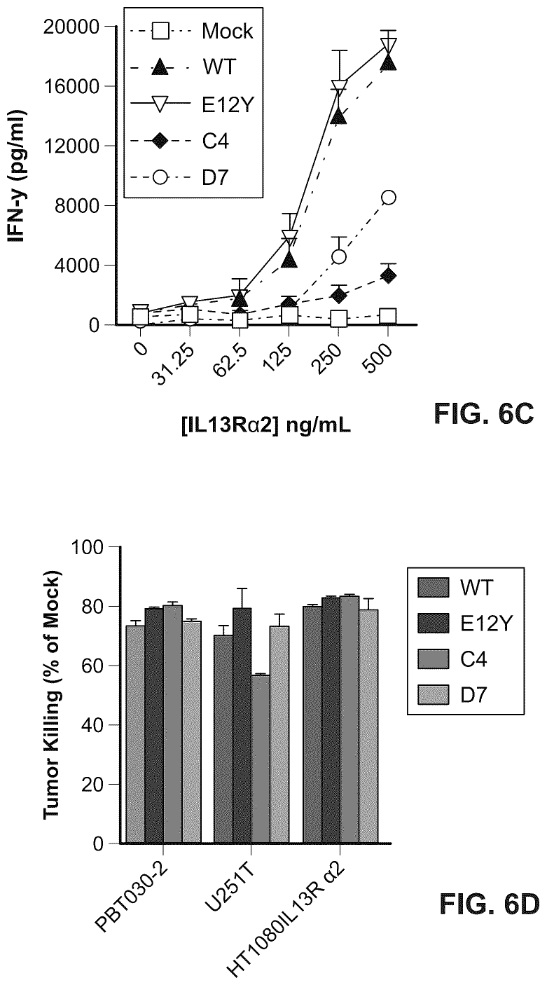

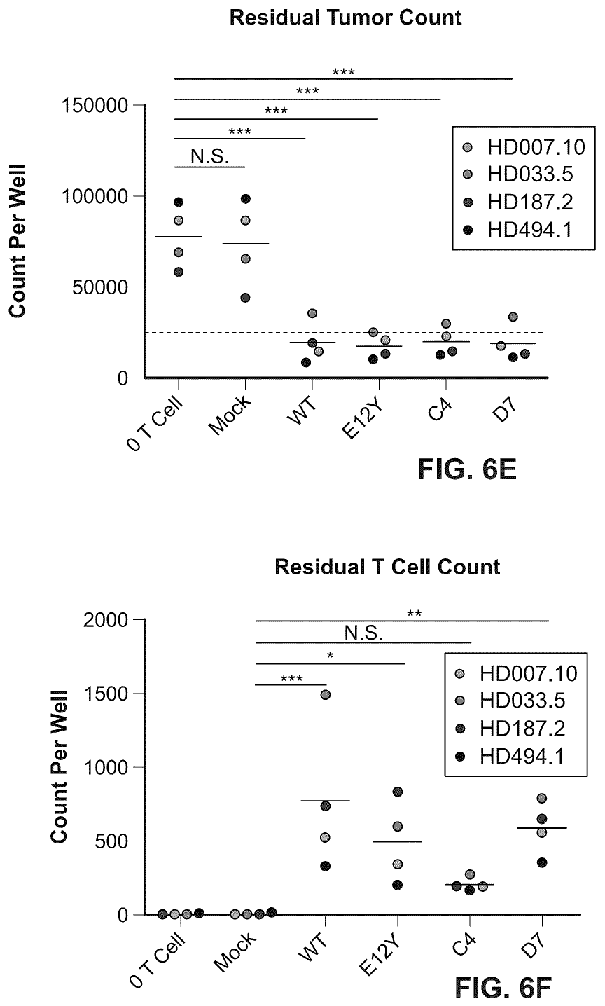

We assessed IL13Rα2-targeting abilities of the IL13 wild type and variant CAR T cells by examining antigen-specific T cell activation. For these functional studies we used three IL13Rα2-expressing human cancer cell lines. The patient-derived primary glioblastoma tumor line PBT030-2 and human glioma line U251T endogenously express IL13Rα2 at high levels, consistent with its overexpression in pathological conditions (FIG. 5A-5C). The IL13 receptor family negative human fibrosarcoma cell line HT1080 was engineered to overexpress IL13Rα2 (HT1080-IL13Rα2). IL13Rα2 expression on the cell lines was confirmed by flow cytometry, western blot and qPCR (FIG. 5A-5C). Using CD107a as a marker of degranulation, we evaluated CAR T cell effector function after co-culturing the cells with the three IL13Rα2-expressing cell lines at a 1:1 effector to target (E:T) ratio for five hours. WT, E12Y, D7, and C4 CAR T cells had comparable CD107a expression against all the IL13Rα2-expressing cell lines, measured as percent positive cells, with negligible CD107a expression seen in mock (untransduced) T cells (FIG. 6A). As another measure of T cell activity, we evaluated cytokine production, both intracellular IFN-γ production by flow cytometry (FIG. 6B) and secreted IFN-γ in response to increasing concentrations of immobilized recombinant human IL13Rα2-Fc (FIG. 6C). The percentage of cells positive for intracellular IFN-γ after a 5-hour incubation at E:T 1:1 with the IL13Rα2-expressing cell lines was similar across WT, E12Y, D7, and C4 CAR T cells, with mock T cells producing negligible IFN-γ. The levels of secreted IFN-γ as a function of IL13Rα2 concentration varied across the CAR T cell types, with WT and E12Y CAR T cells secreting the highest IFN-γ concentration, followed by D7, and then C4 CAR T cells.

To further investigate functional differences between IL13 wild type and variant CAR T cells, we performed in vitro tumor killing assays. CAR T cells were co-cultured with tumor targets PBT030-2, U251T, and HT1080-IL13Rα2 at E:T 1:10 for two days, and viable remaining tumor cells were counted by flow cytometry with results expressed as percentage normalized to the tumor cell count after incubation with mock T cells. All of the CAR T cells (WT, E12Y, D7, and C4) killed the tumor cells with similar efficiency (FIG. 6D). The functional characteristics of the CAR T cells were consistent across four distinct healthy donors within each experiment. In an extended long-term killing assay, CAR T cells co-cultured at E:T 1:50 with PBT030-2 had similar cytolytic activity regardless of IL13 variant and across four donors (FIG. 6E). Importantly, cytotoxicity observed using IL13 mutein CARs was significantly higher than mock and no T cell controls (p<0.005 in all cases). From the same experiments, analysis of live CAR T cells after seven days of co-culture showed persistence/proliferation in the wild type and variant CAR T cells, compared with the no T cell and mock controls, in which no live T cells were observed (FIG. 6F). Of note, C4 displayed lower levels of CAR T cell persistence/expansion as compared to the other IL13 CARs, suggesting reduced potential for antigen-dependent proliferation.

### Example 5

Patient-derived IL13Rα2+Xenograft Mouse Models Demonstrate Improved Survival after Treatment with IL13 Variant CAR T cells

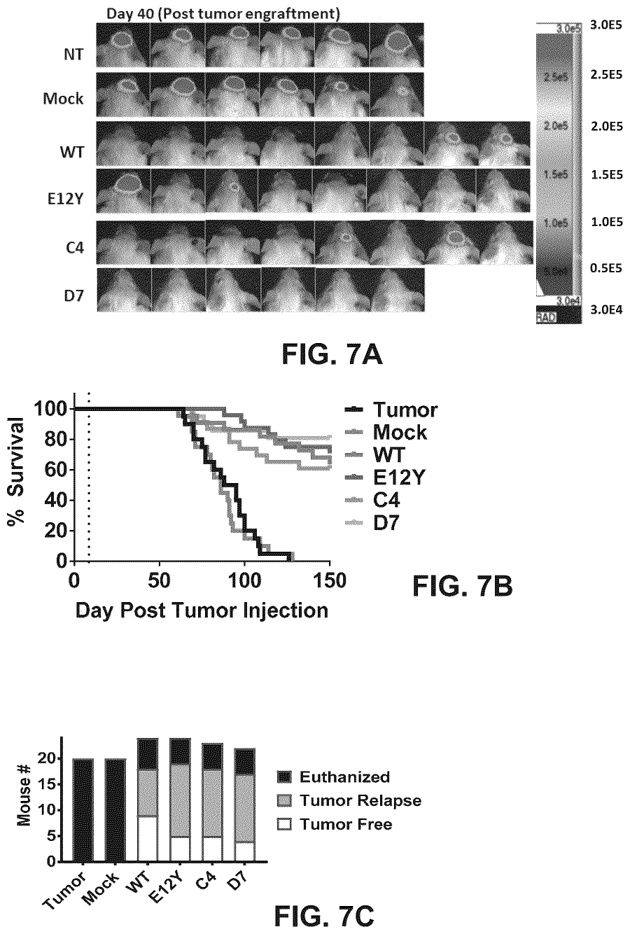

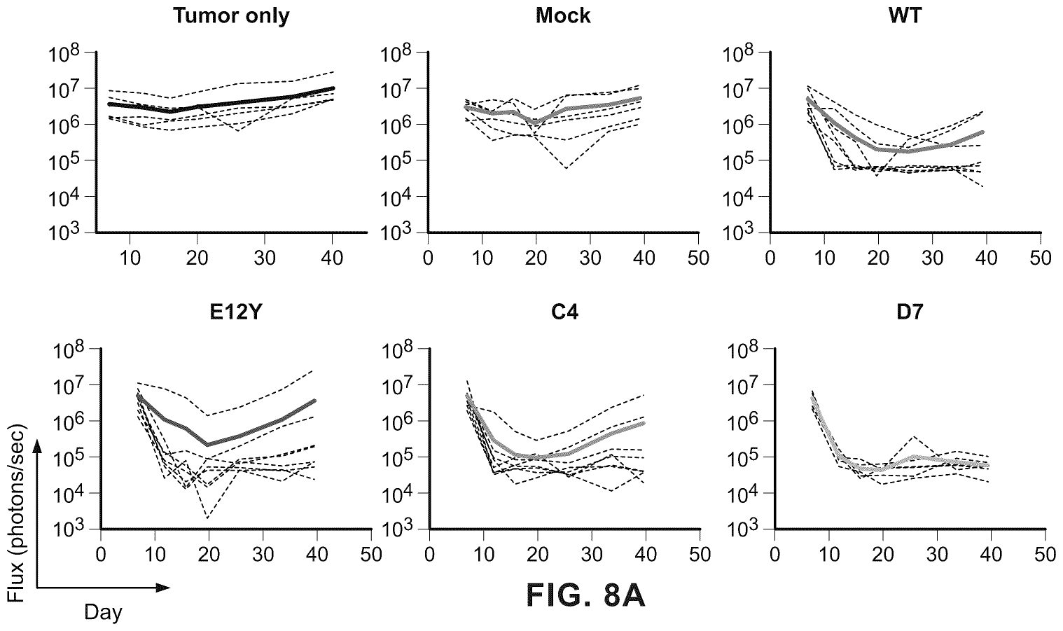

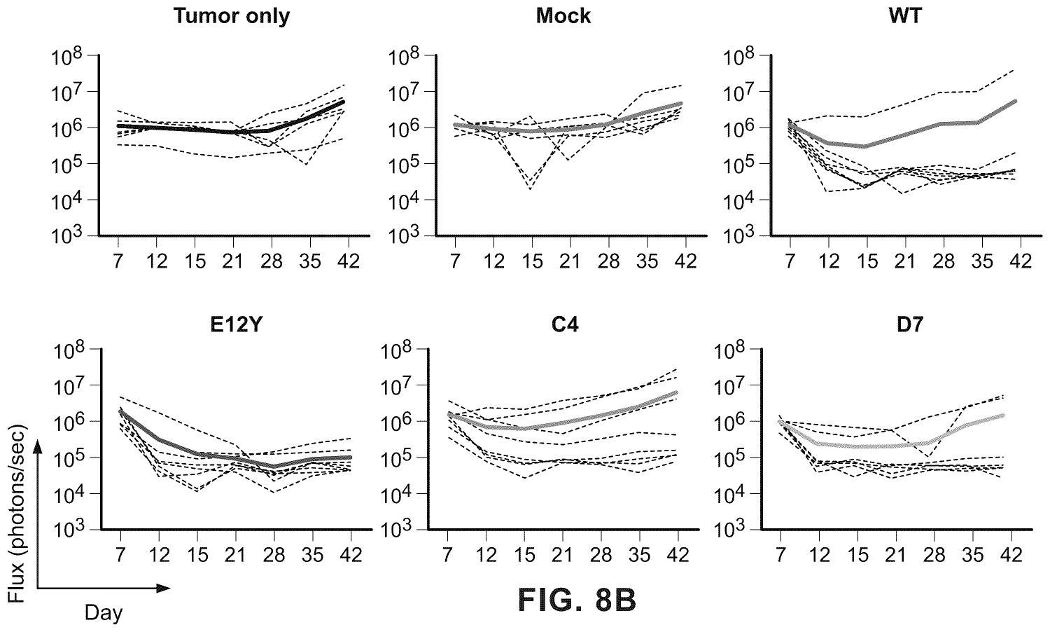

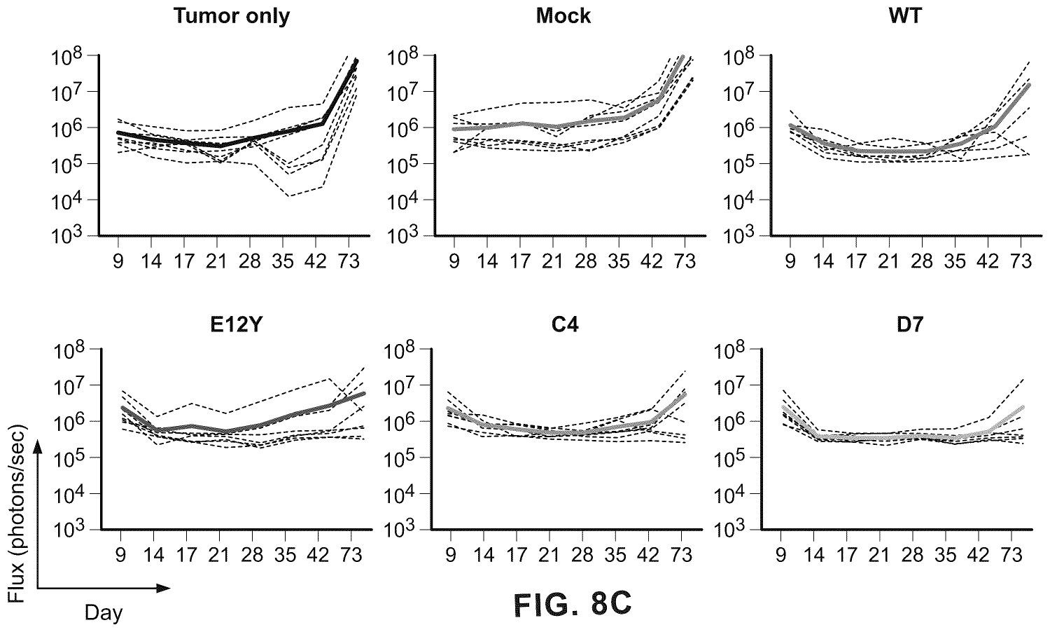

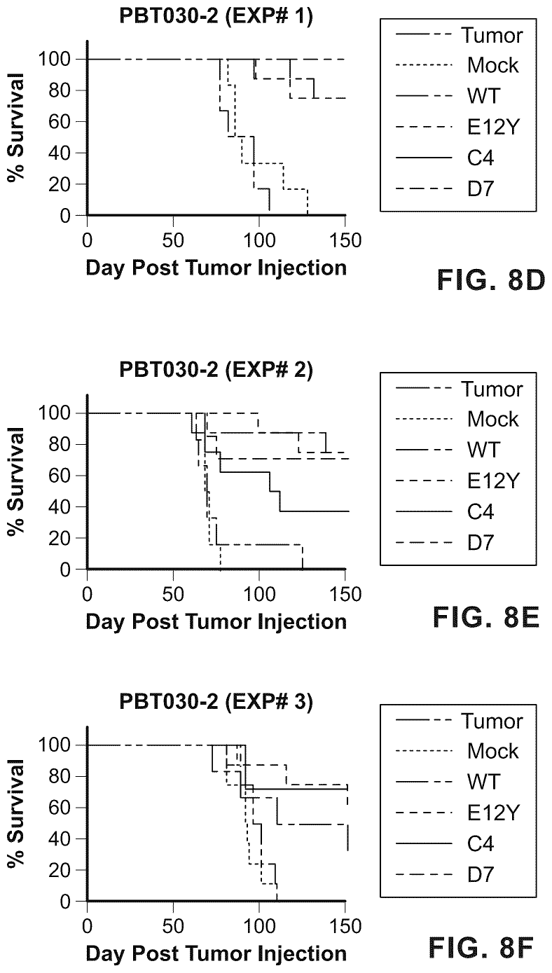

We evaluated the in vivo anti-tumor efficacy of the CAR T cells in our previously established xenograft brain tumor model with IL13Rα2 PBT030-2 cells engineered to express the firefly luciferase (ffluc) reporter gene (Brown, Starr et al. 2012). In three independent experiments, tumor-bearing NSG mice (1×10⁶ tumor cells injected intracranially; 9±1 days engraftment) that received intratumoral (i.t.) injection of 0.3×10⁶ mock (untransduced) T cells exhibited tumor growth and survival similar to non-treated controls, whereas treatment with WT, E12Y, C4, and D7 CAR T cells efficiently reduced tumor burden (FIG. 7A). Kaplan-Meier survival analysis demonstrates improved survival for mice treated with IL13 wild type and variant CAR T cells (FIG. 7B and FIG. 8), and by day 150 the mice treated IL13 wild type and variant CAR T cells had similar numbers of tumor-free and tumor relapsed mice (FIG. 7C). Taken together, these results demonstrate that C4 and D7 CAR T cells exhibit similar in vivo anti-tumor activity for IL-13Rα2 expressing tumors compared with WT and E12Y CAR T cell treatment.

### Example 6

### IL13 Variant CAR T Cells Display Diminished Effector Activity Against IL13Rα1-Expressing Tumors

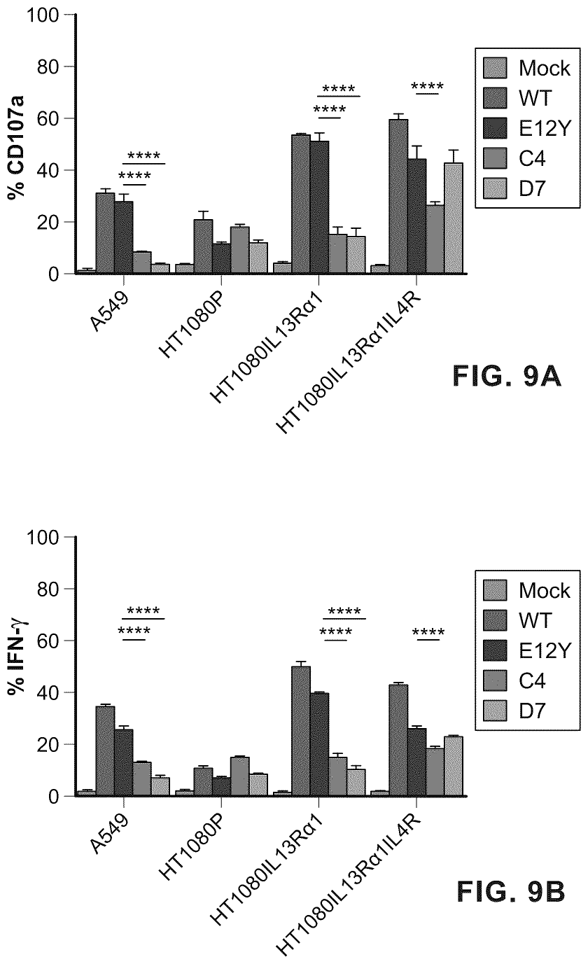

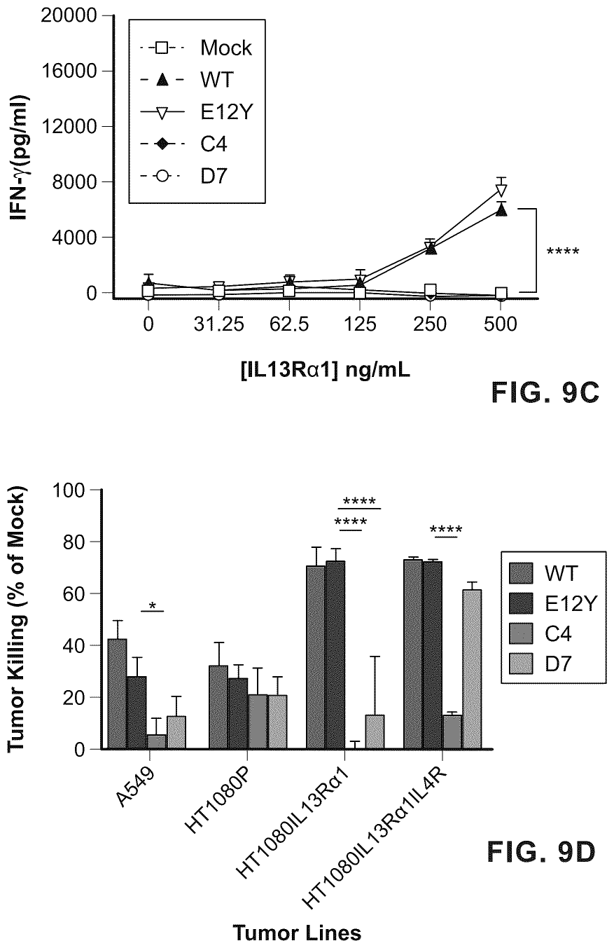

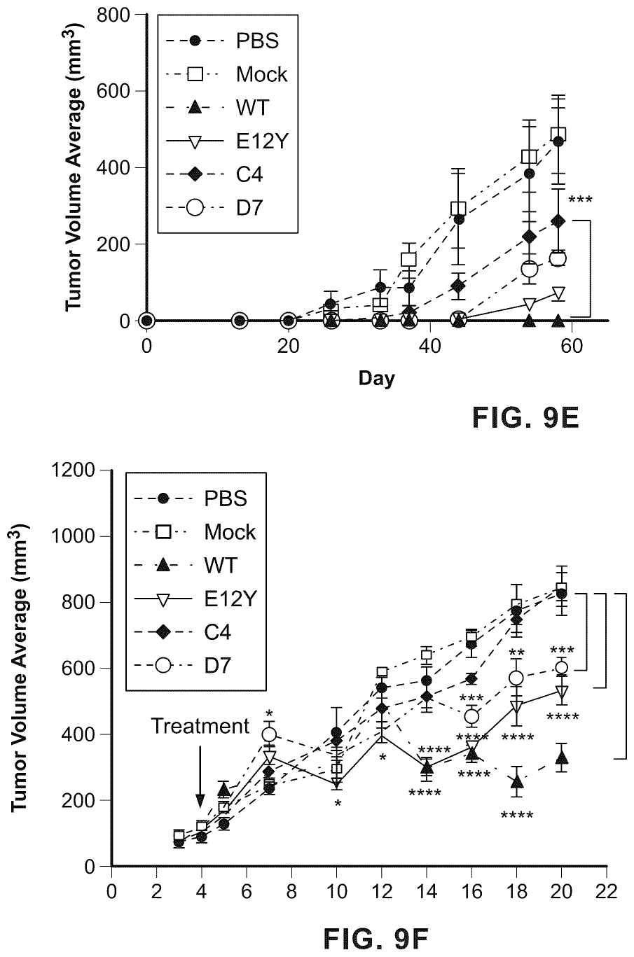

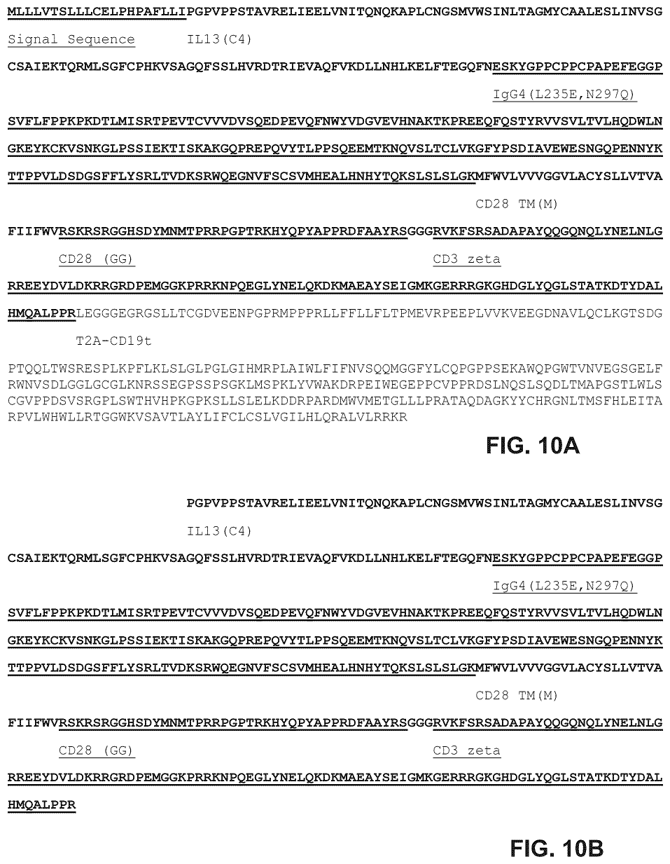

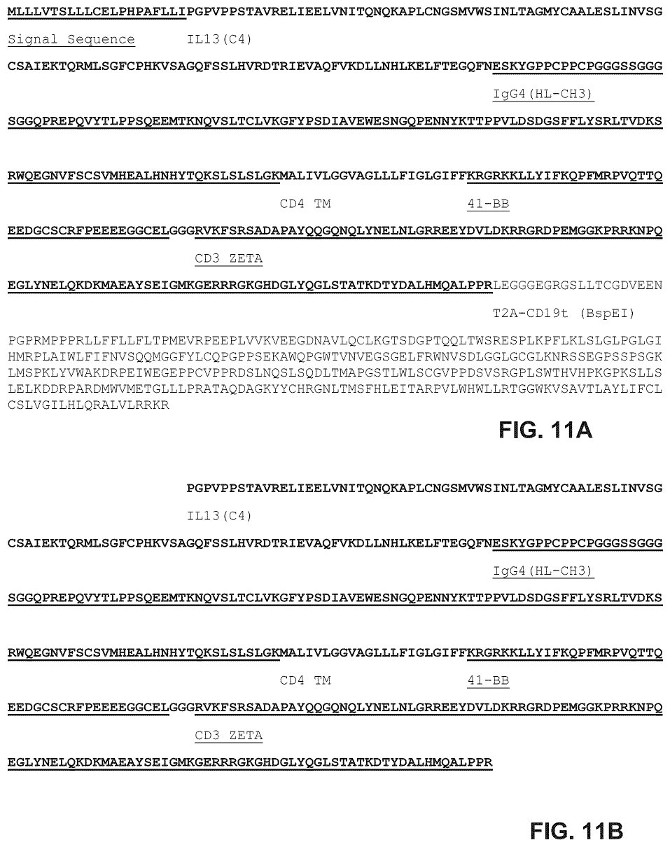

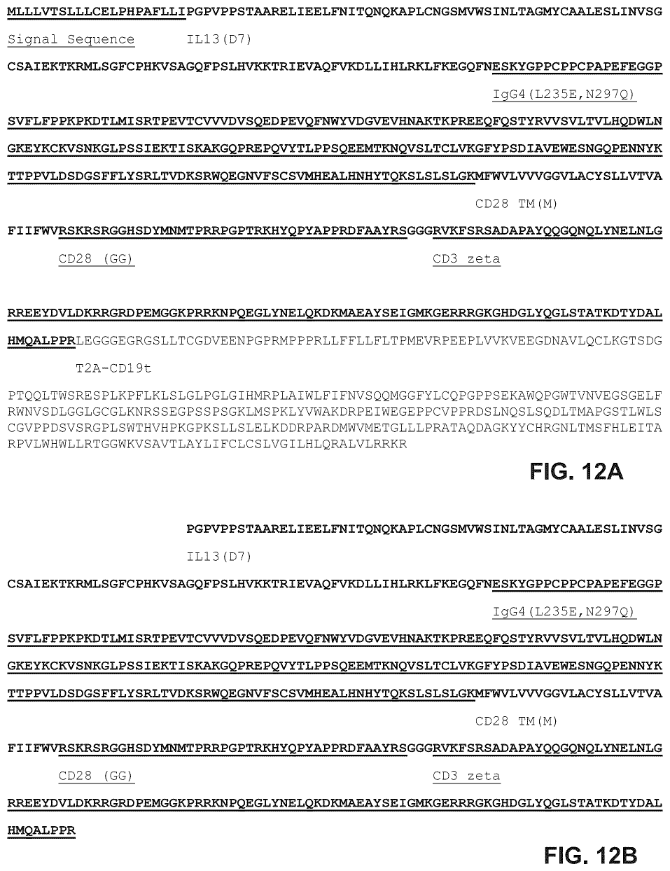

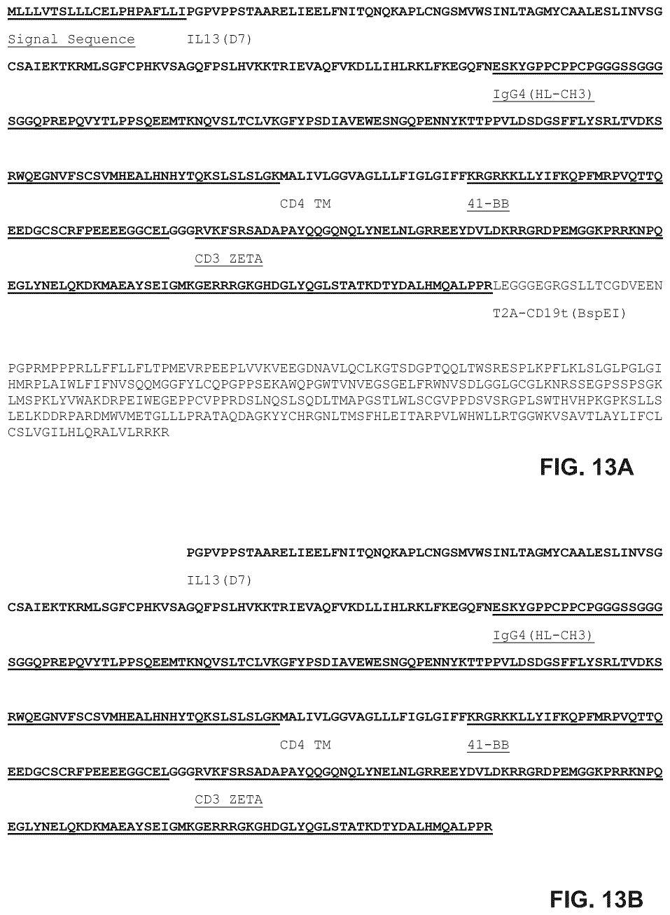

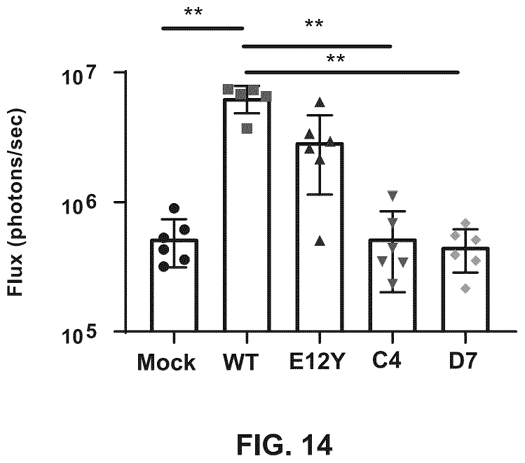

Several human cancer cell lines with varying expression of IL13Rα1 were used to evaluate relative effector activity of IL13 wild type and variant CAR T cells. Human lung adenocarcinoma cell line A549 endogenously expresses moderate levels of IL13Rα1 with no detectable IL13Rα2 or IL4Rα. Human fibrosarcoma cell line HT1080, which does not express IL13Rα1, IL13Rα2, or IL4Rα, was engineered to overexpress either IL13Rα1(denoted HT1080-IL13Rα1) or both IL13Rα1 and IL4Rα (denoted HT1080-IL13Rα1-IL4Rα) (FIG. 5A-5C). In 5 hour co-culture of CAR T cells with IL13Rα1-expressing tumor cells (A549 and HT1080-IL13Rα1) at 1:1 E:T, WT and E12Y induced significantly more surface expression of CD107a (FIG. 9A) and intracellular expression of IFN-γ (FIG. 9B) than C4 and D7 (p<0.05 for all comparisons). In contrast, when CAR T cells were co-cultured with HT1080-IL13Rα1-IL4Rα, E12Y and D7 showed similar expression of surface CD107a and intracellular IFN-γ, whereas C4 displayed attenuated response relative to E12Y in both assays (p<0.05 for both). Notably, against HT1080-IL13Rα1-IL4Rα cells, all the mutein CAR T cells (E12Y, C4, and D7) had a significantly lower frequency of IFN-γ producing cells compared with WT CAR T cells. Evaluation of secreted IFN-γ after culture with plate-bound IL13Rα1 revealed distinct differences in T cell activity among the CAR variants. WT and E12Y both secreted significantly higher IFN-γ levels compared with C4 and D7, which had nearly undetectable IFN-γ (FIG. 9C). The results of long-term killing assays against IL13Rα1-expressing cancer cell lines mirrored those of the degranulation and cytokine production assays: C4 and D7 CAR T cells showed diminished killing relative to WT and E12Y (FIG. 9D). Against HT1080-IL13Rα1-IL4Rα, D7 killed comparably to E12Y and WT whereas C4 showed significantly less tumor killing (p<0.05 for all comparisons).

To interrogate the selectivity of the C4 and D7 mutein CAR T cells in vivo, we investigated the anti-tumor activity of the CAR T cells in xenograft models using IL13Rα1-expressing tumor cells. In order to detect small differences in CAR T cell activity with greater sensitivity, we used the Winn assay to directly evaluate effector activity by incubating tumor and T cells together for two hours prior to injection (Winn 1960, Winn 1961). We co-cultured 1×10⁶ WT, E12Y, C4 and D7 CAR T cells with 0.1×10⁶ A549 cells for 2 hours followed by engraftment of the co-cultured cells into NSG mice (FIG. 9E). Following tumor growth kinetics over 60 days, the WT CAR T cells ablated engraftment of IL13Rα1-expressing tumors. All CAR T cell variants displayed some delay in engraftment relative to PBS and mock T cell treated tumors. At later time points, C4 CAR T cells displayed significantly less growth inhibition than WT and E12Y (p<0.05 for all comparisons on days 54 and 58).

To evaluate the CAR T cell variants against tumors expressing the high affinity pair IL13Rα1/IL4Rα, NSG mice were xenotransplanted subcutaneously with 0.5×106 HT1080-IL13Rα1-IL4Rα with 4-day engraftment. The mice were treated with mock, WT, E12Y, C4, and D7 CAR T cells, or PBS by intratumoral injection (FIG. 9F). Again, the C4 CAR T cells had the least amount of antitumor activity, with tumor growth comparable to PBS treated and mock T cell treated mice (FIG. 9F). Similar to the in vitro killing assays, D7 and E12Y CAR T cell treatment showed comparable decreased antitumor activity relative to WT CAR T cells (p<0.05 for all comparisons Day 16 and thereafter).

### Methods Used in Examples

Tumor lines

PBT030-2 is a patient derived primary glioblastoma tumor sphere line which was heterotopically passaged twice in NOD/Scid IL2RγCnull (NSG) mice (Brown, Starr et al. 2012). (Brown, Starr et al. 2012). Established human tumor lines A549 (lung carcinoma), and HT1080 (fibrosarcoma) were obtained from the American Tissue Culture Collection (ATCC) and maintained in DMEM (Gibco, Grand Island, NY) supplemented with 10% FBS, 2 mM L-glutamine, and 25 mM HEPES. HT-1080 was modified lentivirally to express IL13Rα1, both IL13Rα1 and IL4Rα, or IL13Rα2. U251T glioma line was a gift from Dr. Waldemar Debinski and grown as previously described (Brown, Warden et al. 2013). Cell line TF-1 (erythroleukemia) was grown in RPMI containing 10% FBS, penicillin-streptomycin, 2 mM L-glutamine and GM-CSF to promote proliferation and survival. All cell lines were maintained at 37° C. with 5% CO2.

Flow cytometry

Intracellular phospho-STAT6 staining was performed with pSTAT6-Alexa488 (BD Biosciences, 1:50) after ice-cold methanol (100% v/v) permeabilization. The induction of STAT6 phosphorylation was calculated by subtracting the Mean Fluorescence Intensity (MFI) of the stimulated samples from that of the unstimulated sample. The normalized values were plotted against cytokine concentration to yield dose-response curves from which the EC50 values were calculated based on nonlinear least squares regression fit to a sigmoidal curve.

CAR expression was assessed using biotinylated anti-Fc (Jackson ImmunoResearch, West Gove, PA1: 100) antibody followed by streptavidin-PE (BD Bioscience, San Jose, CA, 1:20) and by staining for the truncated CD19 extracellular sequence with CD19-PE-Cy7 (BD Bioscience, cl, SJ25C1, 1:100). Target lines were characterized by staining with IL13Rα2-PE (Biolegend, cl. SHM38, 1:100), IL13Rα1 (Biolegend, cl. SS12B, 1:100), and IL-4Rα-PE (BD Pharmingen, cl. hIL4R-M57, 1:20). In other assays, additional antibodies were used as specified: CD107a-FITC (BD Biosciences, cl. H4A3, 1:9), CD45 PerCP (BD Biosciences, cl. 2D1, 1:20), CD3-VioBlue (Milentyi Biotec, Inc, 1:20), CD8 APC-Cy7 (BD Biosciences, cl. SK1, 1:50), and IFNγ-APC (BD Biosciences, cl. B27, 1:100). Cells were For staining, cells were washed and re-suspended in FACS Stain Solution (HBSS, 20% v/v FBS, 0.1% w/v NaN3), incubated with antibodies for 30 min at 4° C., followed by secondary stain if necessary, then washed and run on the MACSQuant (Miltenyi Biotec, Bergisch Gladbach, Germany) Flow data was analyzed with FBS Express 4 (De Novo Software, Los Angeles, CA).

### Protein Expression and Purification

Human IL-13 and the IL-13 variants were cloned into the pAcGP67-A vector (BD Biosciences) in frame with an N-terminal gp67 signal sequence and a C-terminal hexahistidine tag and produced using the baculovirus expression system, as described in (LaPorte, Juo et al. 2008). Baculovirus stocks were prepared by transfection and amplification in Spodoptera frugiperda (Sf9) cells grown in SF900II media (Invitrogen) and protein expression was carried out in suspension Trichoplusiani (High Five) cells grown in InsectXpress media (Lonza). Following expression, proteins were captured from High Five supernatants after 48 h by nickel-NTA agarose (Qiagen) affinity chromatography, concentrated, and purified by size exclusion chromatography on a Superdex 200 column (GE

Healthcare), equilibrated in 10 mM HEPES (pH 7.2) containing 150 mM NaCl. Recombinant cytokines were purified to greater than 98% homogeneity. For biotinylated receptor expression, IL-13Rα1/IL-13Rα2 ectodomains were cloned into the pAcGP67-A vector with a C-terminal biotin acceptor peptide (BAP)-LNDIFEAQKIEWHW followed by a hexahistidine tag. Receptors were coexpressed with BirA ligase in the presence of excess biotin (10 μM). Protein concentrations were quantified by UV spectroscopy at 280 nm using a Nanodrop2000 spectrometer (Thermo Scientific).

Yeast display of IL-13

General yeast display methodologies are modified from previously described protocols (Boder and Wittrup 1997). Human IL-13 cDNA was cloned into the yeast display vector pCT302. S. cerevisiae strain EBY100 was transformed with the pCT302_IL-4 vector and grown for two days at 30° C. on SDCAA plates. Individual colonies of IL-13-displaying yeast were grown overnight at 30° C. in SDCAA liquid media (pH 4.5), followed by induction in SGCAA media (pH 4.5) for 2 days at 20° C. Yeast were stained with biotinylated IL-13Rα1 or IL-13Rα2 followed by incubation with streptavidin couple to Alexa-647 dye. Fluorescence was analyzed on an Accuri C6 flow cytometer.

### Surface Plasmon Resonance

SPR experiments were conducted on a Biacore T100 instrument using a Biacore SA sensor chip (GE Healthcare). Biotinylated IL-13Rα1/IL-13Rα2 was captured at a low density (50-100 response units (RU)) and kinetics measurements were conducted at 30 μL/min. An unrelated biotinylated protein was immobilized as a reference surface for the SA sensor chip with matching RU to the experimental surface. All measurements were made using 3-fold serial dilutions of IL-13 agonists in the running buffer (1×HBS-P, 0.1% BSA). The IL-13Rα1/IL-13Rα2 bound to the chip surface was regenerated with 7 mM glycine (pH 3.0) and 250 mM NaCl. Kinetic parameters were determined using 120 s to 190 s of IL-13 agonist association time and 20 s to 1200 s dissociation time. All data fitting was performed using the Biacore T100 evaluation software version 2.0 with a 1:1 Langmuir binding model.

### TF-1 Cell Proliferation Assays

Two thousand TF-1 cells/well were seeded in a 96 well plate and stimulated with the indicated doses of IL-13 wt and the selected IL-13 agonists. After 96 h of stimulation, cells were harvested and cell number was determined using flow cytometry-based counting on an Accuri C6 flow cytometer. The number of cells obtained for each agonist was plotted against the cytokine concentration in order to obtain sigmoidal dose/response curves, from which the TF-1 proliferation EC50 values were calculated.

### CAR Constructs

The codon-optimized IL-13 (E13Y) variant CAR sequence was previously described (Brown, Badie et al. 2015). The ribosomal skip T2A sequence (Donnelly, Luke et al. 2001) was fused by PCR splice overlap extension to the truncated CD19t sequence obtained from the leader peptide to the transmembrane spanning components (i.e., base pairs 1-972) of a CD19-containing plasmid. The IL13-variant and T2A-CD19t fragments were ligated into the previously described epHIV7 lentiviral vector (Wang, Naranjo et al. 2012). The CD28 costimulatory sequence was then inserted by splice overlap PCR, and then that construct underwent sequential site directed mutagenesis using the QuikChange II XL kit (Agilent Technologies, Santa Clara, CA) to generate the CAR variants.

### Isolation of Enriched Tn/mem Cells

Blood products were obtained from healthy donors under protocols approved by the City of Hope (COH) Internal Review Board. Peripheral blood mononuclear cells (PBMCs) were isolated by density gradient centrifugation over Ficoll-Paque (GE Healthcare, Little Chalfont, UK). PBMCs were incubated with clinical-grade anti-CD25 and anti-CD14 microbeads (Miltenyi Biotec, Bergisch Gladbach, Germany) for 30 min at room temperature (RT) in XVivo15 media (BioWhittaker, Walkersville, MD) containing 10% fetal calf serum (FBS)

(HyClone, GE Healthcare). CD25+ and CD14+ cells were then immediately depleted using the CliniMACS depletion mode according to the manufacturer's instructions (Miltenyi Biotec). After centrifugation, the unlabeled negative fraction of cells was resuspended in CliniMACS PBS/EDTA buffer (Miltenyi Biotec) containing 0.5% human serum albumin (HSA) (CSL Behring, King of Prussia, PA) and then labeled with clinical grade biotinylated-DREG56 monoclonal antibody (mAb) (City of Hope Center for Biomedicine and Genetics) at 0.1 μg/10⁶ cells for 30 min at RT. The cells were then washed and resuspended in a final volume of 100 mL CliniMACS PBS/EDTA containing 0.5% HSA. After 30 min incubation with 1.25 mL anti-biotin microbeads (Miltenyi Biotec), the CD62L+ fraction (Tn/mem) was purified with positive selection on CliniMACS according to the manufacturer's instructions and resuspended in X-Vivo15 media containing 10% FBS.

Activation, Lentiviral Transduction, and ex vivo Expansion of CAR T Cells

Tn/mem cells were stimulated with Dynabeads Human T expander CD3/CD28 (Invitrogen, Carlsbad, CA) at a 1:3 cell to bead ratio and transduced with lentivirus at an multiplicity of infection of 1.5-3 in X-Vivo15 containing 10% FBS (Hyclone Laboratories, Logan, UT) and 100 μg/mL protamine sulfate (APP Pharmaceuticals, Schaumburg, IL), 50 U/mL recombinant human (rh) IL-2, and 0.5 ng/mL rhIL-15. Cultures were then maintained at 37° C., 5% CO2, with addition of X-Vivo15 media (10% FBS) as required to keep cell density around 6×10⁵ cells/mL, with cytokine supplementation 3 times a week. On day 7 of culture, the CD3/CD28 Dynabeads were removed from cultures using the DynaMag 5 magnet (Invitrogen). T cell lines were enriched with EasySep™ CD19 selection kit II (Stemcell, Cambridge, MA) around day 14 and propagated for 19-24 days prior to cryopreservation.

### Cytokine Production Assays

For degranulation and intracellular IFN-γ assessment, CAR T cells and tumors were co-cultured at a 1:1 effector to target ratio in X-Vivo15 media without cytokines. CD107a and Golgi Stop (BD Biosciences, 1:1500 v/v) were added to the co-culture prior to the 5 hour incubation at 37° C. Subsequently, the intact cells were stained with human CD45, CD3, CD8, CD19 and IL13Rα2 antibodies. The cells were then fixed, permeabilized using Cytofix/Cytoperm (BD Biosciences) per manufacturer's instructions, stained for IFN-γ and analyzed.

For ELISAs, T cells were cultured overnight at 5×10³ effector per well on flat bottom 96-well plates that had been coated with 500, 250, 125, 62.5 or 31.25 ng/well rhIL13Rα1-Fc chimera or IL13Rα2-Fc chimera (R&D Systems, Minneapolis, MN). Supernatants were then evaluated for IFN-γ levels using the Legend Max ELISA kit (BioLegend, San Diego, CA) per manufacturer's instructions.

### Cytotoxicity Assays

T cells and tumors were co-cultured at 1:10 effector to target ratio in X-Vivo15 media without the addition of cytokines in 96-well plates for 2 days. For extended killing assays, effectors and targets were co-cultured at 1:50 ratio for 7 days in the absence of cytokines, with fresh media replenishment every 3-4 days. At the end of assay, adherent tumors were harvest enzymatically using trypsin (Corning, Corning, NY). Cells were then stained with human CD45, CD8, CD19 and IL13Rα2 and assessed by flow. Tumor killing by CAR T cells was calculated by comparing viable CD45-negative cell counts relative to that observed with mock (non-transduced) T cells.

### Xenograft Models

All mouse experiments were approved by the COH Institute Animal Care and Use Committee. In orthotopic model, ffLuc+ PBT030-2 cells (1×10⁵) were stereotactically implanted into the right forebrain of NSG mice on day 0. Mice were then treated intratumorally with 0.2-2.0×106 CAR T cells as indicated for each experiment. Groups of mice were then monitored for tumor engraftment by Xenogen non-invasive optical imaging as previously described (Kahlon, Brown et al. 2004), or for survival, with euthanasia applied according to the American Veterinary Medical Association Guidelines. Subcutaneous model was established by injecting HT1080 tumors (5×105, 50 μl) in 50% v/v Matrigel® (corning, corning, NY) to the flank of NSG mice. Four days after tumor engraftment, CAR T cells (5×10⁶) were injected intratumorally and tumors sizes were monitored by caliper. To perform Winn assay, A549 tumors (1×10⁵) and CAR T cells (1×10⁶) were co-incubated in culture media at 37° C. for 2 hours. Cell mixtures were then mix with 50% v/v Matrigel® and injected to the flank of NSG mice.

### Statistical Analysis

Statistical significance was determined by Student t-test (two groups), one-way ANOVA (>3 groups, Bonferroni adjustment) or Log-rank (Kaplan-Meir survival curve, Mantel-Cox adjustment) in GraphPad Prism (GraphPad Software, San Diego, CA). Significance levels are represented as *, P<0.05; **, P<0.01; ***, P<0.001; ns indicates not significant.

Beard, R. E., D. Abate-Daga, S. F. Rosati, Z. Zheng, J. R. Wunderlich, S. A. Rosenberg and R. A. Morgan (2013). “Gene expression profiling using nanostring digital RNA counting to identify potential target antigens for melanoma immunotherapy.” Clin Cancer Res 19 (18): 4941-4950.

Boder, E. T. and K. D. Wittrup (1997). “Yeast surface display for screening combinatorial polypeptide libraries.” Nat Biotechnol 15 (6): 553-557.

Boyiadzis, M. M., M. V. Dhodapkar, R. J. Brentjens, J. N. Kochenderfer, S. S. Neelapu, M. V. Maus, D. L. Porter, D. G. Maloney, S. A. Grupp, C. L. Mackall, C. H. June and M. R. Bishop (2018). “Chimeric antigen receptor (CAR) T therapies for the treatment of hematologic malignancies: clinical perspective and significance.” J Immunother Cancer 6 (1): 137.

Brombacher, T. M., J. K. Nono, K. S. De Gouveia, N. Makena, M. Darby, J. Womersley, O. Tamgue and F. Brombacher (2017). “IL-13-Mediated Regulation of Learning and Memory.” J Immunol 198 (7): 2681-2688.

Brown, C. E., D. Alizadeh, R. Starr, L. Weng, J. R. Wagner, A. Naranjo, J. R. Ostberg, M. S. Blanchard, J. Kilpatrick, J. Simpson, A. Kurien, S. J. Priceman, X. Wang, T. L. Harshbarger, M. D'Apuzzo, J. A. Ressler, M. C. Jensen, M. E. Barish, M. Chen, J. Portnow, S. J. Forman and B. Badie (2016). “Regression of Glioblastoma after Chimeric Antigen Receptor T-Cell Therapy.” N Engl J Med 375 (26): 2561-2569.

Brown, C. E., B. Badie, M. E. Barish, L. Weng, J. R. Ostberg, W. C. Chang, A. Naranjo, R. Starr, J. Wagner, C. Wright, Y. Zhai, J. R. Bading, J. A. Ressler, J. Portnow, M. D'Apuzzo, S. J. Forman and M. C. Jensen (2015). “Bioactivity and Safety of IL13Rα1pha2-Redirected Chimeric Antigen Receptor CD8+ T Cells in Patients with Recurrent Glioblastoma.” Clin Cancer Res 21 (18): 4062-4072.

Brown, C. E., R. Starr, B. Aguilar, A. F. Shami, C. Martinez, M. D′Apuzzo, M. E. Barish, S. J. Forman and M. C. Jensen (2012). “Stem-like tumor-initiating cells isolated from IL13Rα1pha2 expressing gliomas are targeted and killed by IL13-zetakine-redirected T Cells.” Clin Cancer Res 18 (8): 2199-2209.

Brown, C. E., C. D. Warden, R. Starr, X. Deng, B. Badie, Y. C. Yuan, S. J. Forman and M. E. Barish (2013). “Glioma IL13Rα1pha2 is associated with mesenchymal signature gene expression and poor patient prognosis.” PLOS One 8 (10): e77769.

Chiu, M. L. and G. L. Gilliland (2016). “Engineering antibody therapeutics.” Curr Opin Struct Biol 38:163-173.

Debinski, W. and D. M. Gibo (2000). “Molecular Expression Analysis of Restrictive Receptor for Interleukin 13, a Brain Tumor-associated Cancer/Testis Antigen.” Molecular Medicine 6 (5): 440-449.

Donnelly, M. L., G. Luke, A. Mehrotra, X. Li, L. E. Hughes, D. Gani and M. D. Ryan (2001). “Analysis of the aphthovirus 2A/2B polyprotein ‘cleavage’ mechanism indicates not a proteolytic reaction, but a novel translational effect: a putative ribosomal ‘skip’.” J Gen Virol 82 (Pt 5): 1013-1025.

Grupp, S. A., M. Kalos, D. Barrett, R. Aplenc, D. L. Porter, S. R. Rheingold, D. T. Teachey, A. Chew, B. Hauck, J. F. Wright, M. C. Milone, B. L. Levine and C. H. June (2013). “Chimeric antigen receptor-modified T cells for acute lymphoid leukemia.” N Engl J Med 368 (16): 1509-1518.

Hecker, M., Z. Zaslona, G. Kwapiszewska, G. Niess, A. Zakrzewicz, E. Hergenreider, J. Wilhelm, L. M. Marsh, D. Sedding, W. Klepetko, J. Lohmeyer, S. Dimmeler, W. Seeger, N. Weissmann, R. T. Schermuly, N. Kneidinger, O. Eickelberg and R. E. Morty (2010). “Dysregulation of the IL-13 receptor system: a novel pathomechanism in pulmonary arterial hypertension.” Am J Respir Crit Care Med 182 (6): 805-818.

Hinck, A. P. (2018). “Structure-guided engineering of TGF-betas for the development of novel inhibitors and probing mechanism.” Bioorg Med Chem 26 (19): 5239-5246.

Jonnalagadda, M., A. Mardiros, R. Urak, X. Wang, L. J. Hoffman, A. Bernanke, W. C. Chang, W. Bretzlaff, R. Starr, S. Priceman, J. R. Ostberg, S. J. Forman and C. E. Brown (2015).

“Chimeric antigen receptors with mutated IgG4 Fc spacer avoid fc receptor binding and improve T cell persistence and antitumor efficacy.” Mol Ther 23 (4): 757-768.

Kahlon, K. S., C. Brown, L. J. Cooper, A. Raubitschek, S. J. Forman and M. C. Jensen (2004). “Specific recognition and killing of glioblastoma multiforme by interleukin 13-zetakine redirected cytolytic T cells.” Cancer Res 64 (24): 9160-9166.

Kahlon, K. S., C. Brown, L. J. N. Cooper, A. Raubitschek, S. J. Forman and M. C. Jensen (2004). “Specific Recognition and Killing of Glioblastoma Multiforme by Interleukin 13-Zetakine Redirected Cytolytic T Cells.” Cancer Res 64:9160-9166.

Kioi, M., M. Kawakami, T. Shimamura, S. R. Husain and R. K. Puri (2006). “Interleukin-13 receptor alpha2 chain: a potential biomarker and molecular target for ovarian cancer therapy.” Cancer 107 (6): 1407-1418.

Kong, S., S. Sengupta, B. Tyler, A. J. Bais, Q. Ma, S. Doucette, J. Zhou, A. Sahin, B. S. Carter, H. Brem, R. P. Junghans and P. Sampath (2012). “Suppression of human glioma xenografts with second-generation IL13R-specific chimeric antigen receptor-modified T cells.” Clin Cancer Res 18 (21): 5949-5960.

Krebs, S., K. K. Chow, Z. Yi, T. Rodriguez-Cruz, M. Hegde, C. Gerken, N. Ahmed and S. Gottschalk (2014). “T cells redirected to interleukin-13Ralpha2 with interleukin-13 mutein-chimeric antigen receptors have anti-glioma activity but also recognize interleukin-13Ralpha1.” Cytotherapy 16 (8): 1121-1131.

LaPorte, S. L., Z. S. Juo, J. Vaclavikova, L. A. Colf, X. Qi, N. M. Heller, A. D. Keegan and K. C. Garcia (2008). “Molecular and structural basis of cytokine receptor pleiotropy in the interleukin-4/13 system.” Cell 132 (2): 259-272.

Lee, L., B. Draper, N. Chaplin, B. Philip, M. Chin, D. Galas-Filipowicz, S. Onuoha, S. Thomas, V. Baldan, R. Bughda, P. Maciocia, E. Kokalaki, M. P. Neves, D. Patel, M. Rodriguez-Justo, J. Francis, K. Yong and M. Pule (2018). “An APRIL-based chimeric antigen receptor for dual targeting of BCMA and TACI in multiple myeloma.” Blood 131 (7): 746-758.

Long, K. B., R. M. Young, A. C. Boesteanu, M. M. Davis, J. J. Melenhorst, S. F. Lacey, D. A. DeGaramo, B. L. Levine and J. A. Fraietta (2018). “CAR T Cell Therapy of Non-hematopoietic Malignancies: Detours on the Road to Clinical Success.” Frontiers in Immunology 9.

Lupardus, P. J., M. E. Birnbaum and K. C. Garcia (2010). “Molecular Basis for Shared Cytokine Recognition Revealed in the Structure of an Unusually High Affinity Complex between IL-13 and IL-13Rα2.” Structure 18 (3): 332-342.

Maude, S. L., N. Frey, P. A. Shaw, R. Aplenc, D. M. Barrett, N. J. Bunin, A. Chew, V. E. Gonzalez, Z. Zheng, S. F. Lacey, Y. D. Mahnke, J. J. Melenhorst, S. R. Rheingold, A. Shen, D. T. Teachey, B. L. Levine, C. H. June, D. L. Porter and S. A. Grupp (2014). “Chimeric antigen receptor T cells for sustained remissions in leukemia.” N Engl J Med 371 (16): 1507-1517.

Moraga, I., D. Richter, S. Wilmes, H. Winkelmann, K. Jude, C. Thomas, M. M. Suhoski, E. G. Engleman, J. Piehler and K. C. Garcia (2015). “Instructive roles for cytokine-receptor binding parameters in determining signaling and functional potency.” Science Signaling 8 (402).

Murata, T., J. Taguchi and R. K. Puri (1998). “Interleukin-13 Receptor Alpha' but Not Alpha Chain: A Functional Component of Interleukin-4 Receptors.” Blood 91:3884-3891.

Papageorgis, P., S. Ozturk, A. W. Lambert, C. M. Neophytou, A. Tzatsos, C. K. Wong, S. Thiagalingam and A. I. Constantinou (2015). “Targeting IL13Rα1pha2 activates STAT6-TP63 pathway to suppress breast cancer lung metastasis.” Breast Cancer Res 17:98.

Porter, D. L., B. L. Levine, M. Kalos, A. Bagg and C. H. June (2011). “Chimeric Antigen Receptor-Modified T Cells in Chronic Lymphoid Leukemia.” The New England Journal of Medicine 365 (8): 725-733.

Rosenfeld, L., M. Heyne, J. M. Shifman and N. Papo (2016). “Protein Engineering by Combined Computational and In Vitro Evolution Approaches.” Trends Biochem Sci 41 (5): 421-433.

Shaffer, D. R., B. Savoldo, Z. Yi, K. K. Chow, S. Kakarla, D. M. Spencer, G. Dotti, M. F. Wu, H. Liu, S. Kenney and S. Gottschalk (2011). “T cells redirected against CD70 for the immunotherapy of CD70-positive malignancies.” Blood 117 (16): 4304-4314.

Shaffer, D. R., P. Zhou and S. Gottschalk (2014). “Foreign or Domestic CARs: Receptor Ligands as Antigen-Binding Domains.” Medical Sciences 2:23-36.

Shibasaki, N., T. Yamasaki, T. Kanno, R. Arakaki, H. Sakamoto, N. Utsunomiya, T. Inoue, T. Tsuruyama, E. Nakamura, O. Ogawa and T. Kamba (2015). “Role of IL13RA2 in Sunitinib Resistance in Clear Cell Renal Cell Carcinoma.” PLOS One 10 (6): e0130980.

Shimamura, T., T. Fujisawa, S. R. Husain, B. Joshi and R. K. Puri (2010). “Interleukin 13 mediates signal transduction through interleukin 13 receptor alpha2 in pancreatic ductal adenocarcinoma: role of IL-13 Pseudomonas exotoxin in pancreatic cancer therapy.” Clin Cancer Res 16 (2): 577-586.

Sockolosky, J. T., E. Trotta, G. Parisi, L. Picton, L. L. Su, A. C. Le, A. Chhabra, S. L. Silveria, B. M. George, I. C. King, M. R. Tiffany, K. Jude, L. V. Sibener, D. Baker, J. A. Shizuru, A. Ribas, J. A. Bluestone and K. C. Garcia (2018). “Selective targeting of engineered T cells using orthogonal IL-2 cytokine-receptor complexes.” Science 359:1037-1042.

Thaci, B., C. E. Brown, E. Binello, K. Werbaneth, P. Sampath and S. Sengupta (2014). “Significance of interleukin-13 receptor alpha 2-targeted glioblastoma therapy.” Neuro-Oncology 16 (10): 1304-1312.

Wang, X., A. Naranjo, C. E. Brown, C. Bautista, C. W. Wong, W. C. Chang, B. Aguilar, J. R. Ostberg, S. R. Riddell, S. J. Forman and M. C. Jensen (2012). “Phenotypic and functional attributes of lentivirus-modified CD19-specific human CD8+ central memory T cells manufactured at clinical scale.” J Immunother 35 (9): 689-701.

Winn, H. J. (1960). “Immune mechanisms in homotransplantation. I. The role of serum antibody and complement in the neutralization of lymphoma cells.” J Immunol 84:530-538.

Winn, H. J. (1961). “Immune mechanisms in homotransplantation. II. Quantitative assay of the immunologic activity of lymphoid cells stimulated by tumor homografts.” J Immunol 86: 228-239.

Xie, M., X. Wu, J. Zhang and C. He (2015). “IL-13 receptor a2 is a negative prognostic factor in human lung cancer and stimulates lung cancer growth in mice.” Oncotarget 6 (32): 32902-32913.

Zhang, T., B. A. Lemoi and C. L. Sentman (2005). “Chimeric NK-receptor-bearing T cells mediate antitumor immunotherapy.” Blood 106 (5): 1544-1551.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims. All references are herein incorporated in their entirety for any and all purposes.

What is claimed is:

1. A nucleic molecule comprising a nucleotide sequence encoding a chimeric antigen receptor (CAR), wherein the chimeric antigen receptor comprises:
a targeting domain comprising or consisting of the amino acid sequence of SEQ ID NO: 26 or 27;
a spacer domain comprising or consisting of the amino acid sequence of SEQ ID NO: 9, 10, or 11;
a transmembrane domain comprising or consisting of the amino acid sequence of SEQ ID NO: 14, 15, or 16;
a co-stimulatory domain comprising or consisting of the amino acid sequence of SEQ ID NO: 22, 23, or 24; and
a CD3ζ domain comprising or consisting of the amino acid sequence of SEQ ID NO: 21.

2. The nucleic acid molecule of claim 1, wherein the targeting domain consists of the amino acid sequence of SEQ ID NO: 26 or 27.

3. The nucleic acid molecule of claim 1, wherein the chimeric antigen receptor comprises:
(i) a targeting domain comprising or consisting of the amino acid sequence of SEQ ID NO: 26; a spacer domain comprising or consisting of the amino acid sequence of SEQ ID NO: 11; a transmembrane domain comprising or consisting of the amino acid sequence of SEQ ID NO: 15; a co-stimulatory domain comprising or consisting of the amino acid sequence of SEQ ID NO: 23; and a CD3ζ domain comprising or consisting of the amino acid sequence of SEQ ID NO: 21;
(ii) a targeting domain comprising or consisting of the amino acid sequence of SEQ ID NO: 26; a spacer domain comprising or consisting of the amino acid sequence of SEQ ID NO: 9; a transmembrane domain comprising or consisting of the amino acid sequence of SEQ ID NO: 16; a co-stimulatory domain comprising or consisting of the amino acid sequence of SEQ ID NO: 24; and a CD3ζ domain comprising or consisting of the amino acid sequence of SEQ ID NO: 21;
(iii) a targeting domain comprising or consisting of the amino acid sequence of SEQ ID NO: 27; a spacer domain comprising or consisting of the amino acid sequence of SEQ ID NO: 11; a transmembrane domain comprising or consisting of the amino acid sequence of SEQ ID NO: 15; a co-stimulatory domain comprising or consisting of the amino acid sequence of SEQ ID NO: 23; and a CD3ζ domain comprising or consisting of the amino acid sequence of SEQ ID NO: 21; or (iv) a targeting domain comprising or consisting of the amino acid sequence of SEQ ID NO: 27; a spacer domain comprising or consisting of the amino acid sequence of SEQ ID NO: 9; a transmembrane domain comprising or consisting of the amino acid sequence of SEQ ID NO: 16; a co-stimulatory domain comprising or consisting of the amino acid sequence of SEQ ID NO: 24; and a CD3ζ domain comprising or consisting of the amino acid sequence of SEQ ID NO: 21.

4. A chimeric antigen receptor comprising:
a targeting domain comprising or consisting of the amino acid sequence of SEQ ID NO: 26 or 27;
a spacer domain comprising or consisting of the amino acid sequence of SEQ ID NO: 9, 10, or 11;
a transmembrane domain comprising or consisting of the amino acid sequence of SEQ ID NO: 14, 15, or 16;
a co-stimulatory domain comprising or consisting of the amino acid sequence of SEQ ID NO: 22, 23, or 24; and
a CD3ζ domain comprising or consisting of the amino acid sequence of SEQ ID NO: 21.

5. The chimeric antigen receptor of claim 4, wherein the targeting domain consists of the amino acid sequence of SEQ ID NO: 26 or 27.

6. The chimeric antigen receptor of claim 4, comprising:
(i) a targeting domain comprising or consisting of the amino acid sequence of SEQ ID NO: 26; a spacer domain comprising or consisting of the amino acid sequence of SEQ ID NO: 11; a transmembrane domain comprising or consisting of the amino acid sequence of SEQ ID NO: 15; a co-stimulatory domain comprising or consisting of the amino acid sequence of SEQ ID NO: 23; and a CD3ζ domain comprising or consisting of the amino acid sequence of SEQ ID NO: 21;
(ii) a targeting domain comprising or consisting of the amino acid sequence of SEQ ID NO: 26; a spacer domain comprising or consisting of the amino acid sequence of SEQ ID NO: 9; a transmembrane domain comprising or consisting of the amino acid sequence of SEQ ID NO: 16; a co-stimulatory domain comprising or consisting of the amino acid sequence of SEQ ID NO: 24; and a CD3ζ domain comprising or consisting of the amino acid sequence of SEQ ID NO: 21;
(iii) a targeting domain comprising or consisting of the amino acid sequence of SEQ ID NO: 27; a spacer domain comprising or consisting of the amino acid sequence of SEQ ID NO: 11; a transmembrane domain comprising or consisting of the amino acid sequence of SEQ ID NO: 15; a co-stimulatory domain comprising or consisting of the amino acid sequence of SEQ ID NO: 23; and a CD3ζ domain comprising or consisting of the amino acid sequence of SEQ ID NO: 21; or
(iv) a targeting domain comprising or consisting of the amino acid sequence of SEQ ID NO: 27; a spacer domain comprising or consisting of the amino acid sequence of SEQ ID NO: 9; a transmembrane domain comprising or consisting of the amino acid sequence of SEQ ID NO: 16; a co-stimulatory domain comprising or consisting of the amino acid sequence of SEQ ID NO: 24; and a CD3ζ domain comprising or consisting of the amino acid sequence of SEQ ID NO: 21.

7. An expression vector comprising the nucleic acid molecule of claim 1.

8. A viral vector comprising the nucleic acid molecule of claim 1.

9. A population of human T cells transduced by a vector comprising the nucleic acid molecule of claim 1.

10. The population of human T cells of claim 9, wherein the population of human T cells comprise central memory T cells, naive memory T cells, pan T cells, or PBMC depleted for CD25+ cells and CD14+ cells.

11. A method of treating a patient suffering from glioblastoma, pancreatic ductal adenocarcinoma, melanoma, ovarian carcinoma, renal cell carcinoma, breast cancer or lung cancer, comprising administering a population of autologous or allogeneic human T cells transduced by a vector comprising the nucleic acid molecule of claim 1.

12. The method of claim 11, wherein the cells are administered locally or systemically.

13. The method of claim 12, wherein the cells are administered by single or repeat dosing.

14. A method of preparing CAR T cells comprising: providing a population of autologous or allogeneic human T cells and transducing the T cells by a vector comprising the nucleic acid molecule of claim 1.

15. A population of human T cells expressing the chimeric antigen receptor of claim 4.

16. The population of human T cells of claim 15, wherein the population of human T cells comprise central memory T cells, naive memory T cells, pan T cells, or PBMC depleted for CD25+ cells and CD14+ cells.

17. A method of treating a patient suffering from glioblastoma, pancreatic ductal adenocarcinoma, melanoma, ovarian carcinoma, renal cell carcinoma, breast cancer or lung cancer, comprising administering the population of human T cells of claim 15.

18. The method of claim 17, wherein the patient is suffering from glioblastoma.

19. The method of claim 17, wherein the population of human T cells are allogenic human T cells.

20. The method of claim 17, wherein the population of human T cells are autologous human T cells.
